(19) United States
(12) Patent Application Publication
(10) Pub. No.: US 2011/0218609 A1
(43) Pub. Date: Sep. 8, 2011

(54) FILL TUBE MANIFOLD AND DELIVERY METHODS FOR ENDOVASCULAR GRAFT

(75) Inventors: Michael V. Chobotov, Santa Rosa, CA (US); Mary Jane Marston, Windsor, CA (US); Mark E, Purter, Windsor, CA (US); Patrick Stephens, Santa Rosa, CA (US); James R. Watson, Santa Rosa, CA (US); Robert G. Whirley, Santa Rosa, CA (US)

(73) Assignee: TRIVASCULAR, INC., Santa Rosa, CA (US)

(21) Appl. No.: 13/024,255

(22) Filed: Feb. 9, 2011

Related U.S. Application Data
(60) Provisional application No. 61/303,295, filed on Feb. 10, 2010.

(51) Int. Cl. A61F 2/84; A61F 2/82

(52) U.S. Cl. 623/1.11; 623/1.2

(57) ABSTRACT

Some embodiments relate in part to endovascular prostheses and methods of deploying same. Embodiments may be directed more specifically to inflatable stent grafts and methods of positioning and deploying such devices within the body of a patient. Some embodiments include inflation devices and methods that allow an inflatable portion of an inflatable stent graft to be inflated from a desired location within the inflatable portion.

### FIELD OF THE INVENTION

Some embodiments relate in part to endovascular prostheses and methods of deploying same. Embodiments may be directed more specifically to stent grafts and methods of making and deploying same within the body of a patient.

### BACKGROUND

An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta which is a serious and life threatening condition for which intervention is generally indicated. Existing methods of treating aneurysms include invasive surgical procedures with graft replacement of the affected vessel or body lumen or reinforcement of the vessel with a graft.

Surgical procedures to treat aortic aneurysms can have relatively high morbidity and mortality rates due to the risk factors inherent to surgical repair of this disease as well as long hospital stays and painful recoveries. This is especially true for surgical repair of TAAs, which is generally regarded as involving higher risk and more difficulty when compared to surgical repair of AAAs. An example of a surgical procedure involving repair of a AAA is described in a book titled Surgical Treatment of Aortic Aneurysms by Denton A. Cooley, M.D., published in 1986 by W. B. Saunders Company.

Due to the inherent risks and complexities of surgical repair of aortic aneurysms, endovascular repair has become a widely-used alternative therapy, most notably in treating AAAs. Early work in this field is exemplified by Lawrence, Jr. et al. in “Percutaneous Endovascular Graft: Experimental Evaluation”, Radiology (May 1987) and by Mirich et al. in “Percutaneously Placed Endovascular Grafts for Aortic Aneurysms: Feasibility Study,” Radiology (March 1989). Commercially available endoprostheses for the endovascular treatment of AAAs include the AneuRx® stent graft manufactured by Medtronic, Inc. of Minneapolis, Minn., the Zenith® stent graft system sold by Cook, Inc. of Bloomington, Ind., the PowerLink® stent graft system manufactured by Endologix, Inc. of Irvine, Calif., and the Excluder® stent graft system manufactured by W.L. Gore & Associates, Inc. of Newark, Del. A commercially available stent graft for the treatment of TAAs is the TAG™ system manufactured by W.L. Gore & Associates, Inc.

When deploying devices by catheter or other suitable instrument, it is advantageous to have a flexible and low profile stent graft and delivery system for passage through the various guiding catheters as well as the patient's sometimes tortuous anatomy. Many of the existing endovascular devices and methods for treatment of aneurysms, while representing significant advancement over previous devices and methods, use systems having relatively large transverse profiles, often up to 24 French. Also, such existing systems have greater than desired lateral stiffness, which can complicate the delivery process. In addition, the sizing of stent grafts may be important to achieve a favorable clinical result. In order to properly size a stent graft, the treating facility typically must maintain a large and expensive inventory of stent grafts in order to accommodate the varied sizes of patient vessels due to varied patient sizes and vessel morphologies. Alternatively, intervention may be delayed while awaiting custom size stent grafts to be manufactured and sent to the treating facility. As such, minimally invasive endovascular treatment of aneurysms is not available for many patients that would benefit from such a procedure and can be more difficult to carry out for those patients for whom the procedure is indicated.

What have been needed are stent graft systems and methods that are adaptable to a wide range of patient anatomies and that can be safely and reliably deployed using a flexible low profile system.

### SUMMARY

Some embodiments are directed to a method of deploying an inflatable endovascular stent graft. The method may include advancing a delivery catheter that includes the endovascular stent graft in a radially constrained state to a deployment site within a patient's vasculature. The endovascular graft may then be partially deployed so as to allow at least a portion of a proximal self-expanding member of the endovascular graft to radially expand. An imaging system is aligned relative to the patient's body such that an imaging axis of the imaging system is substantially orthogonal to a longitudinal axis of a tubular main body portion of the endovascular stent graft. The partially deployed endovascular graft is positioned in an axial direction to a desired position within the patient's vasculature and the proximal self-expanding member of the endovascular graft fully deployed so as to engage an interior luminal surface within the patient's vasculature.

Some embodiments of a method of deploying an inflatable endovascular stent graft include advancing a delivery catheter that includes the endovascular stent graft in a radially constrained state to a deployment site within a patient's vasculature. The endovascular graft may then be partially deployed so as to allow at least a portion of a proximal self-expanding member of the endovascular graft to radially expand. An imaging system is aligned relative to the patient's body such that an imaging axis of the imaging system is substantially orthogonal to a longitudinal axis of a tubular main body portion of the endovascular stent graft. The partially deployed endovascular graft is positioned in an axial direction to a desired position within the patient's vasculature and the proximal self-expanding member of the endovascular graft fully deployed so as to engage an interior luminal surface within the patient's vasculature. An inflatable portion of the endovascular stent graft may then be inflated with a fill material.

Some embodiments of an endovascular stent graft include a tubular flexible main body portion and a proximal self-expanding stent member. The stent graft also includes a plurality of radiopaque markers circumferentially disposed about a tubular portion of the endovascular stent graft and lying in a plane that is substantially orthogonal to a longitudinal axis of the tubular main body portion.

Some embodiments of an inflatable endovascular stent graft including a tubular flexible main body portion, a proximal self-expanding stent member and a proximal inflatable cuff. The stent graft also includes a plurality of radiopaque markers circumferentially disposed about a tubular portion of the endovascular stent graft and lying in a plane that is substantially orthogonal to a longitudinal axis of the tubular main body portion.

Some embodiments of a method of deploying an inflatable endovascular stent graft include advancing a delivery catheter that includes the endovascular stent graft in a radially constrained state to a deployment site within a patient's vasculature. The delivery catheter may then be rotated about a longitudinal axis of the delivery catheter until a longitudinal inflatable channel of an inflatable portion of the endovascular stent graft that extends longitudinally along a main body portion of the stent graft is disposed along a greater curve of a vascular lumen of the patient's vasculature within which the delivery system is disposed. The stent graft is then deployed at the deployment site with the longitudinal inflatable channel disposed along the greater curve of the vascular lumen and inflating an inflatable portion including the longitudinal inflatable channel of the endovascular stent graft.

Some embodiments of a method of deploying an inflatable endovascular stent graft include advancing a delivery catheter that includes the endovascular stent graft in a radially constrained state to a deployment site within a patient's vasculature. The delivery catheter is then rotated about a longitudinal axis of the delivery catheter until a longitudinal inflatable channel of the endovascular stent graft that extends longitudinally along a main body portion of the stent graft is disposed along a greater curve of a vascular lumen of the patient's vasculature within which the delivery system is disposed. The endovascular stent graft may then be partially deployed so as to allow at least a portion of a proximal self-expanding member of the endovascular graft to radially expand. The partially deployed endovascular graft may then be positioned in an axial direction to a desired position within the patient's vasculature. The self-expanding member of the endovascular graft is then fully deployed so as to allow the proximal self-expanding member of the endovascular graft to expand and engage an inner luminal surface of the patient's vasculature. An inflatable portion of the endovascular stent graft including the longitudinal inflatable channel is then inflated with a fill material.

Some embodiments of an endovascular stent graft include a flexible main graft body portion including a proximal end, a distal end, and an inflatable portion including at least one longitudinal inflation channel. The stent graft also includes a self-expanding stent member secured to the main graft body portion and one or more radiopaque markers configured to distinguish circumferential rotational position of the at least one longitudinal inflation channel prior to being filled with fill material.

Some embodiments of a method of deploying an inflatable endovascular stent graft advancing a delivery catheter that includes the endovascular stent graft in a radially constrained state to a deployment site within a patient's vasculature with a proximal end of the stent graft disposed towards a flow of blood within the patient's vasculature. The stent graft in the constrained state is axially positioned relative to the deployment site and a proximal self-expanding member of the endovascular graft deployed to expand and engage an interior luminal surface the patient's vasculature. A distal end of the stent graft is then positioned in an axial direction until a tubular main body portion of the stent graft achieves a desired configuration and a distal self-expanding member deployed so as to allow the distal self-expanding member to expand and engage an interior luminal surface of the patient's vasculature.

Some embodiments of a method of deploying an inflatable endovascular stent graft include advancing a delivery catheter that includes the endovascular stent graft in a radially constrained state to a deployment site within a patient's vasculature with a proximal end of the stent graft disposed towards a flow of blood within the patient's vasculature. The endovascular graft may then be partially deployed so as to allow at least a portion of a proximal self-expanding member of the endovascular graft to radially expand. An imaging system is aligned relative to the patient's body such that an imaging axis of the imaging system is substantially orthogonal to a longitudinal axis of a tubular main body portion of the endovascular stent graft. The partially deployed endovascular graft is then positioned in an axial direction to a desired position within the patient's vasculature. The proximal self-expanding member of the endovascular graft may then be fully deployed so as to allow the proximal self-expanding member to expand and engage an interior luminal surface the patient's vasculature. An inflatable portion of the endovascular stent graft is inflated with a fill material. A distal end of the stent graft is positioned in an axial orientation until a tubular main body portion of the stent graft achieves a desired configuration and a distal self-expanding member deployed so as to allow the distal self-expanding member to expand and engage an interior luminal surface of the patient's vasculature.

Some embodiments of a method of deploying an inflatable endovascular stent graft include advancing a delivery catheter that includes the inflatable endovascular stent graft in a radially constrained state to a deployment site within a patient's vasculature. The delivery catheter may also be advanced with a proximal end of the stent graft disposed towards a flow of blood within the patient's vasculature. A proximal self-expanding member of the endovascular graft may then be deployed so as to allow the proximal self-expanding member to expand and engage an interior luminal surface the patient's vasculature. An interior volume of an inflatable portion of the endovascular stent graft may be at least partially inflated from a desired location within an interior volume of the inflatable portion with a fill material. A distal end of the stent graft is axially positioned such that a tubular main body portion of the stent graft achieves a desired deployed configuration. A distal self-expanding member of the stent graft may then be deployed so as to allow the distal self-expanding member to expand and engage an interior luminal surface of the patient's vasculature.

Some embodiments of an inflatable endovascular stent graft include at least one self-expanding stent member and a flexible graft body portion secured to the self-expanding member, the graft body portion including a proximal end, a distal end and an inflatable portion. The inflatable stent graft also includes an inflation conduit disposed within the inflatable portion, the inflation conduit including a distal end with an inflation port in fluid communication with an exterior portion of the graft body portion and extending from the distal end into an interior volume of the inflatable portion. The inflation conduit also includes at least one outlet port disposed at a desired position or desired positions within the inflatable portion and configured to first fill the inflatable portion from the desired position or positions.

Some embodiments of a method of deploying an inflatable endovascular stent graft include advancing a delivery catheter that includes the endovascular stent graft in a radially constrained state to a deployment site within a patient's vasculature. The delivery catheter may be advanced with a proximal end of the stent graft disposed towards a flow of blood within the patient's vasculature. A proximal portion of an inflatable portion of the endovascular stent graft may then be inflated with a fill material with the fill material flowing from a proximal portion of the inflatable portion to a distal portion of the inflatable portion.

Some embodiments of an inflatable endovascular stent graft include at least one self-expanding stent member and a flexible graft body portion secured to the self-expanding member. The graft body portion may include at least one tubular portion, a proximal end a distal end and in inflatable portion. An inflation conduit may be disposed within the inflatable portion, the inflation conduit including a distal end with an inflation port in fluid communication with an exterior portion of the graft body portion and extending from the distal end into an interior volume of the inflatable portion.

Some embodiments of an inflatable endovascular stent graft including at least one self-expanding stent member and a flexible graft body portion secured to the self-expanding member. The graft body portion includes at least one tubular portion, a proximal end, a distal end and an inflatable portion including a proximal inflatable cuff disposed at the proximal end of the graft body portion and an inflatable channel extending distally from the proximal inflatable cuff. An inflation conduit is disposed within the inflatable channel. The inflation conduit includes a distal end with an inflation port in fluid communication with an exterior portion of the graft body portion and extending from the distal end through the inflatable channel. The inflation conduit terminates with an outlet port disposed within or near an interior cavity of the proximal inflatable cuff.

Certain embodiments are described further in the following description, examples, claims and drawings.

### BRIEF DESCRIPTION OF THE DRAWINGS

FIG. 1 is an elevation view of an embodiment of an inflatable stent graft.

FIG. 2 is an elevation view in longitudinal section of the stent graft of FIG. 1 as indicated by lines 2-2 in FIG. 3, illustrating an inflation conduit disposed within an inflatable channel of the stent graft.

FIG. 3 is a transverse cross section view of the stent graft of FIG. 1 taken along lines 3-3 of FIG. 1.

FIG. 4 is a transverse cross section view of the stent graft of FIG. 1 taken along lines 4-4 of FIG. 1.

FIG. 5 is an elevation view of an embodiment of an inflation conduit.

FIG. 5A is a transverse cross section view of the inflation conduit of FIG. 5 taken along lines 5A-5A of FIG. 5 and illustrates a bead disposed within a lumen of the inflation conduit.

FIG. 5B is a transverse cross section view of the inflation conduit of FIG. 5 taken along lines 5B-5B of FIG. 5 and illustrates a lumen maintaining bead embodiment disposed within an inner lumen of the inflation conduit.

FIG. 6A is an elevation view of an embodiment of an inflation conduit including a bead disposed within an inner lumen of the inflation conduit.

FIG. 6B shows a top view and a side view of a distal end of a bead having a flattened distal end configured for bonding to an inflation conduit structure.

FIG. 6C is a transverse cross section view of the bead of FIG. 6B taken along lines 6C-6C of the top view of the bead of FIG. 6B.

FIG. 6D is a transverse cross section view of the bead of FIG. 6B taken along lines 6D-6D of the top view of the bead of FIG. 6B.

FIG. 6E is an elevation view in longitudinal section of a junction between tubular members of the inflation conduit embodiment of FIG. 6A indicated by the encircled portion 6E in FIG. 6A and illustrating a flattened distal end of the bead secured in the junction between the tubular members.

FIG. 6F is an elevation view in longitudinal section of the junction between tubular members of the inflation conduit embodiment of FIG. 6A and illustrating a flattened distal end of the bead secured to an inside surface of the tubular member.

FIG. 7 illustrates a delivery system embodiment disposed over a guidewire embodiment within a patient's abdominal aorta and crossing an abdominal aortic aneurysm.

FIG. 8 illustrates the delivery system of FIG. 7 with an outer sheath of the delivery system retracted distally.

FIG. 9 illustrates the delivery system of FIG. 6 with the outer sheath retracted and an embodiment of a proximal self-expanding member of a stent graft embodiment disposed on the delivery system in a state of partial deployment.

FIG. 10 is an enlarged view of a proximal end portion of the stent graft embodiment of FIG. 9 showing the proximal self-expanding member partially deployed.

FIG. 11 is a transverse cross section view of the stent graft of FIG. 10 taken along lines 11-11 of FIG. 10 and illustrating a radiopaque marker configuration of the stent graft embodiment.

FIG. 12A is a perspective view of the stent graft embodiment of FIG. 9 with the proximal self-expanding member partially deployed and illustrating a substantially circular radiopaque marker configuration lying substantially in a plane and indicated by the dashed line of FIG. 12A.

FIG. 12B is an elevation view of the stent graft embodiment of FIG. 12A illustrating an unaligned angle of view of an observer of the stent graft with the angle of view indicated by the arrow between a longitudinal axis of the stent graft and the line of sight of the observer.

FIG. 12C is a schematic view of the radiopaque markers from the unaligned angle of view as depicted in FIG. 12C.

FIG. 13 is a perspective view of a patient on an operating table illustrating spatial adjustment of an imaging system disposed in operative arrangement with the patient.

FIG. 14A shows the stent graft of FIG. 12A aligned to an orthogonal angle of view after spatial adjustment of the imaging system with the radiopaque markers appearing to be linearly aligned as indicated by the dashed line in FIG. 14A.

FIG. 14B shows the orthogonal angle of view of FIG. 14A indicated by the arrow between the longitudinal axis of the stent graft and line of sight of the observer.

FIG. 15 illustrates axial adjustment indicated by the arrow of the delivery system of FIG. 6 with the outer sheath retracted and the embodiment of the proximal self-expanding member of a stent graft embodiment disposed on the delivery system in a state of partial deployment.

FIG. 16 illustrates the stent graft of FIG. 15 with the proximal self-expanding member fully deployed and engaged with a luminal surface of the patient's vasculature and an inflatable portion of the stent graft partially inflated.

FIG. 17 is an enlarged view in section of the stent graft of FIG. 16 indicated by the encircled portion 17 shown in FIG. 16 and illustrating inflation of an inflatable portion of the stent graft by ejection of fill material from the proximal end of an inflation conduit embodiment.

FIG. 17A illustrates inflation of an inflatable portion of the stent graft by ejection of fill material from the proximal end of an inflation conduit embodiment.

FIG. 18 shows the stent graft of FIG. 16 with a flow of blood, indicated by arrows, beginning to fill a lumen of a main body portion of the stent graft embodiment after sealing of the proximal inflatable cuff with a luminal wall of the patient's vasculature.

FIG. 19 shows the stent graft of FIG. 18 more fully filled by a flow of blood.

FIG. 20 illustrates the stent graft of FIG. 19 with the inflatable portion of the stent graft fully filled and the proximal self-expanding member fully deployed.

FIG. 20A illustrates the stent graft of FIG. 20 with a contralateral stent graft extension coupled to a contralateral leg of the stent graft and iliac artery of the patient's vasculature.

FIG. 21 illustrates an embodiment of an inflatable stent graft.

FIG. 22 shows the stent graft of FIG. 21 in section illustrating an inflatable channel and cuffs and an inflation conduit disposed within an interior volume of the inflatable portion.

FIG. 23 is a transverse cross sectional view of the stent graft of FIG. 22 taken along lines 23-23 of FIG. 22 and illustrating the inflation conduit disposed within the inflatable channel of the inflatable portion of the stent graft and a bead disposed within an inner lumen of the inflation conduit.

FIG. 24 illustrates a delivery system embodiment disposed over a guidewire embodiment within a patient's abdominal aorta.

FIG. 25 illustrates the delivery system of FIG. 24 disposed across a thoracic aneurysm and indicating rotational adjustment of the delivery system by an arrow.

FIG. 26 illustrates the delivery system of FIG. 25 with an outer sheath of the delivery system retracted distally and indicating axial adjustment of the delivery system by an arrow.

FIG. 27 illustrates the delivery system of FIG. 26 with a proximal self-expanding member of a stent graft embodiment in a state of partial deployment and further illustrates axial positioning in the partially deployed state as indicated by the arrow.

FIG. 28 is an enlarged view of a proximal end portion of the stent graft embodiment of FIG. 27 showing the proximal self-expanding member partially deployed.

FIG. 29 is a transverse cross section view of the stent graft of FIG. 28 taken along lines 29-29 of FIG. 28 and illustrating a radiopaque marker configuration of the stent graft embodiment.

FIG. 30A is a perspective view of the stent graft embodiment of FIG. 28 with the proximal self-expanding member partially deployed and illustrating a substantially circular radiopaque marker configuration lying substantially in a plane and indicated by the dashed line of FIG. 30A.

FIG. 30B is an elevation view of the stent graft embodiment of FIG. 30A illustrating an unaligned angle of view of an observer of the stent graft with the angle of view indicated by the arrow between a longitudinal axis of the stent graft and the line of sight of the observer.

FIG. 30C is a schematic view of the radiopaque markers from the unaligned angle of view as depicted in FIG. 30C.

FIG. 31A shows the stent graft of FIG. 30A aligned to an orthogonal angle of view after spatial adjustment of the imaging system with the radiopaque markers appearing to be linearly aligned as indicated by the dashed line in FIG. 31A.

FIG. 31B shows the orthogonal angle of view of FIG. 31A indicated by the arrow between the longitudinal axis of the stent graft and line of sight of the observer.

FIG. 32 illustrates axial adjustment of the delivery system of FIG. 25 with the outer sheath retracted and the embodiment of the proximal self-expanding member of a stent graft embodiment disposed on the delivery system in a state of partial deployment.

FIG. 33 illustrates the stent graft of FIG. 32 with the proximal self-expanding member fully deployed and engaged with a luminal surface of the patient's vasculature and an inflatable portion of the stent graft partially inflated.

FIG. 34 shows the stent graft of FIG. 33 with a flow of blood beginning to fill a lumen of a body portion of the stent graft embodiment after sealing of a proximal inflatable cuff with a luminal wall of the patient's vasculature as shown in FIG. 33.

FIG. 35 shows the stent graft of FIG. 34 more fully filled by a flow of blood.

FIG. 36 shows the stent graft of FIG. 35 more fully filled by a flow of blood.

FIG. 37 shows the stent graft of FIG. 36 more fully filled by a flow of blood.

FIG. 38 is an enlarged view of an inflatable portion of the stent graft of FIG. 33 being inflated by ejection of fill material from a proximal end of an inflation conduit embodiment into an interior volume of the inflatable portion of the stent graft.

FIG. 39 illustrates the stent graft of FIG. 19 with the inflatable portion of the stent graft fully filled and the proximal self-expanding member fully deployed.

FIG. 40 illustrates axial adjustment of a distal end of a stent graft disposed within a patient's vasculature after a proximal self-expanding member has been deployed so as to engage a luminal surface of the patient's vasculature.

FIG. 41 illustrates complete deployment of a stent graft with a body portion of the stent graft adjusted towards a least curve of the patient's vasculature.

FIG. 42 illustrates complete deployment of a stent graft with a body portion of the stent graft adjusted towards a greater curve of the patient's vasculature.

The drawings illustrate embodiments of the invention and are not limiting. For clarity and ease of illustration, the drawings are not made to scale and, in some instances, various aspects may be shown exaggerated or enlarged to facilitate an understanding of particular embodiments.

### DETAILED DESCRIPTION

Embodiments may be directed generally to methods and devices for treatment of fluid flow vessels with the body of a patient. Treatment of blood vessels is specifically indicated for some embodiments, and, more specifically, treatment of aneurysms, such as abdominal aortic aneurysms.

Some embodiments of endovascular stent graft assemblies for delivery and deployment within the vasculature of a patient may include non-bifurcated or bifurcated embodiments that include a main graft member formed from a flexible and supple graft material, such as PTFE, having a main fluid flow lumen therein. For some embodiments, flexible graft material including PTFE may include expanded PTFE or ePTFE. The main graft member of bifurcated embodiments may also include an ipsilateral leg with an ipsilateral fluid flow lumen in communication with the main fluid flow lumen, a contralateral leg with a contralateral fluid flow lumen in communication with the main fluid flow lumen and a network of inflatable channels disposed on or otherwise made a part of the main graft member. For some embodiments, the main graft member may have an axial length of about 5 cm to about 10 cm, more specifically, about 7 cm to about 9 cm in order to span an aneurysm of a patient's aorta without engaging the patient's iliac arteries directly with the legs of the main graft member.

The inflatable portion of the stent graft, including inflatable channels of the network of inflatable channels, may be disposed on any portion of the main graft member including the ipsilateral and contralateral legs. The network of inflatable channels may be configured to accept a fill material to provide structural rigidity to the main graft member when the network of inflatable channels is in an inflated state. For some embodiments, the fill or inflation material may be cured, thickened or hardened after it has been disposed within an inflatable portion of the inflatable stent graft. Radiopaque inflation material may be used to facilitate monitoring of the fill process and subsequent engagement of optional graft extensions as well as any other suitable purpose. The network of inflatable channels may include at least one inflatable cuff disposed on a proximal portion, distal portion or any other suitable portion, of the main graft member and may be configured to seal against an inside or luminal surface of a patient's vessel or vasculature, such as the aorta.

A proximal self-expanding anchor member may be disposed at a proximal end of the main graft member and secured to the main graft body or member. An optional distal self-expanding member may also be secured to a distal end of the main body, particularly in non-bifurcated embodiments. The proximal anchor member may have a self-expanding proximal stent portion secured to a self-expanding distal stent portion. The proximal anchor member may be secured to the main graft body member with struts that extend between a connector ring and the distal stent portion. Some embodiments of the struts may have a cross sectional area that is substantially the same as or greater than a cross sectional area of proximal stent portions or distal stent portions adjacent the strut. Such a configuration may be useful in avoiding points of concentrated stress in the proximal anchor member or struts which couple components thereof. For some embodiments, the proximal stent portion of the proximal anchor member further includes a plurality of barbs having sharp tissue engaging tips that are configured to extend in a radial outward direction in a deployed expanded state. For some embodiments, the proximal anchor member includes a 4 crown proximal stent portion and an 8 crown distal stent portion which may be made from a superelastic alloy such as superelastic NiTi alloy.

At least one ipsilateral graft extension having a fluid flow lumen disposed therein may be deployed with the fluid flow lumen of the graft extension sealed to and in fluid communication with a fluid flow lumen of an ipsilateral leg of the main graft member of a bifurcated stent graft embodiment. In addition, at least one contralateral graft extension having a fluid flow lumen disposed therein may be deployed with the fluid flow lumen of the graft extension sealed to and in fluid communication with a fluid flow lumen of a contralateral leg of such a main graft member. For some embodiments, the graft extensions may include an interposed self-expanding stent disposed between at least one outer layer and at least one inner layer of supple layers of graft material. The interposed stent disposed between the outer layer and inner layer of graft material may be formed from an elongate resilient element helically wound with a plurality of longitudinally spaced turns into an open tubular configuration. For some embodiments, the interposed stent is may include a superelastic alloy such as superelastic NiTi alloy. In addition, the graft material of each graft extension may further include at least one axial zone of low permeability for some embodiments.

For some embodiments, an outside surface of a graft extension may be sealed to an inside surface of the respective leg of the main graft and luminal surface of a patient's vasculature when the graft extension is in a deployed state. For some embodiments, the axial length of the ipsilateral and contralateral legs may be sufficient to provide adequate surface area contact with outer surfaces of graft extensions to provide sufficient friction to hold the graft extensions in place. For some embodiments, the ipsilateral and contralateral legs may have an axial length of at least about 2 cm. For some embodiments, the ipsilateral and contralateral legs may have an axial length of about 2 cm to about 6 cm, more specifically, about 3 cm to about 5 cm. The stent graft embodiments discussed herein may include some or all of the features, dimensions or materials as those of the stent graft embodiments discussed in U.S. patent application Ser. No. 12/245,620, filed Oct. 3, 2008, by M. Chobotov et al., titled “Modular Vascular Graft for Low Profile Percutaneous Delivery”, which is incorporated by reference herein in its entirety.

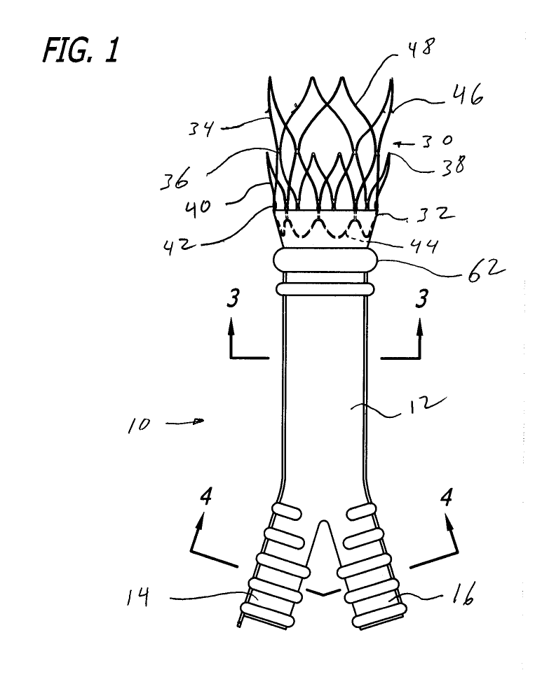

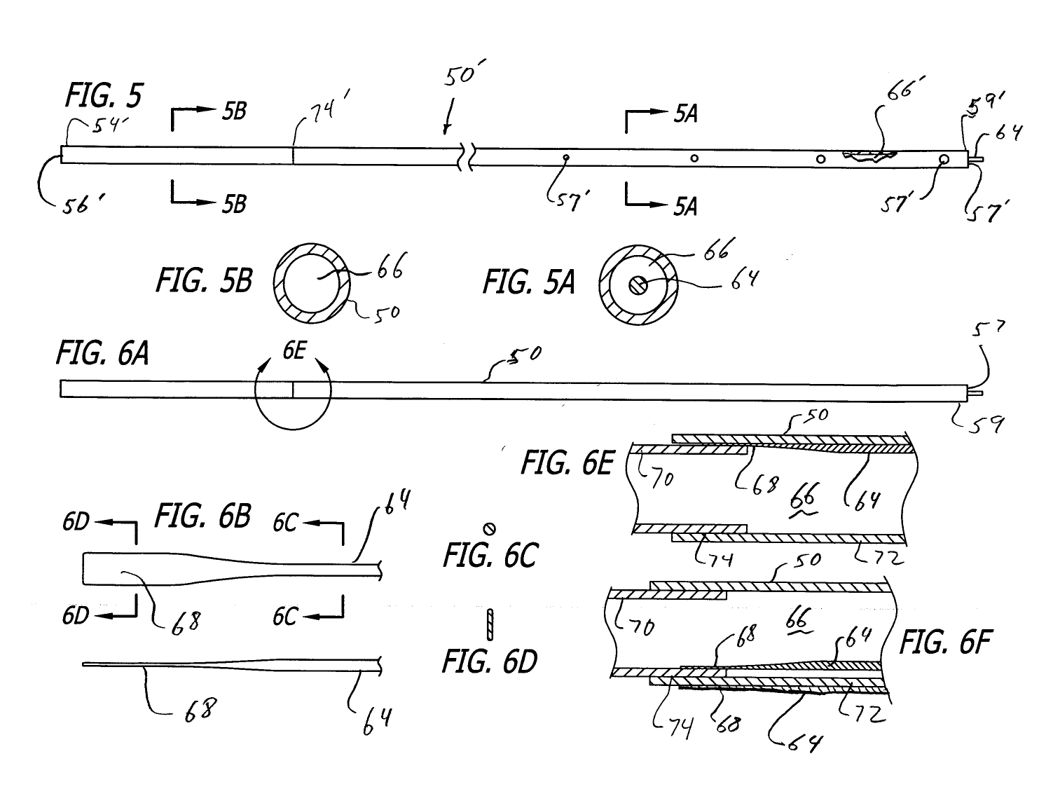

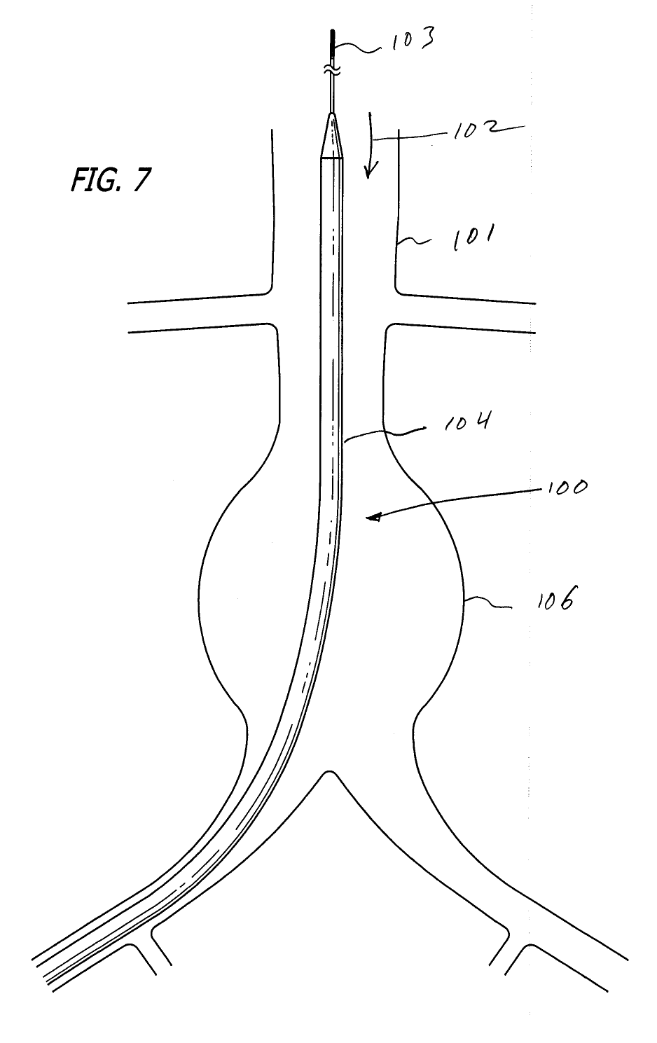

FIGS. 1-4 show a bifurcated embodiment of an inflatable stent graft 10 for treatment of an abdominal aortic aneurysm of a patient. FIGS. 5-6E illustrate embodiments of inflation conduits that may be configured for use within an inflatable portion of an inflatable stent graft to allow the inflatable portion of a stent graft to be inflated from a desired site or sites within an interior volume of the inflatable portion. FIGS. 7-20A illustrate positioning and deployment method embodiments of a bifurcated inflatable stent graft, such as the bifurcated inflatable stent graft embodiment 10 shown in FIGS. 1-4 as well as others. The positioning and deployment method embodiments illustrated in FIGS. 7-20A and discussed herein, may be used to deploy a variety of stent graft embodiments, including inflatable stent graft embodiments 10, in a desired position of a patient's vasculature and in a desired orientation with respect to a patient's vasculature. The illustrated methods may be useful in maintaining control of the deployment process and allow treating personnel to accurately place the end prosthesis while minimizing stresses on the device and the patient's vasculature.

With regard to stent graft embodiments discussed herein, such as stent graft assembly 10, and components thereof, as well as graft extensions and, the term “proximal” refers to a location towards a patient's heart and the term “distal” refers to a location away from the patient's heart. With regard to delivery system catheters and components thereof discussed herein, the term “distal” refers to a location that is disposed away from an operator who is using the catheter and the term “proximal” refers to a location towards the operator.

Referring again to FIGS. 1-4, the inflatable stent graft assembly 10 shown has a bifurcated configuration and includes a graft body portion having a main graft member or body portion 12, an ipsilateral leg 14 and contralateral leg 16. The main graft body portion 12 may have a substantially tubular configuration and has a wall portion 18 that bounds a main fluid flow lumen 20 disposed therein. The ipsilateral leg 14 which may be of a substantially tubular configuration has an ipsilateral port 22 and an ipsilateral fluid flow lumen 24 that is in fluid communication with the main fluid flow lumen 20 and the ipsilateral port 22. The contralateral leg 16 which may be of a substantially tubular configuration has a contralateral port 26 and a contralateral fluid flow lumen 28 that is in fluid communication with the main fluid flow lumen 20 and the contralateral port 26. The main graft 12, ipsilateral leg 14 and contralateral leg 16 form a graft portion having a bifurcated “Y” shaped configuration. The main body portion 12 and legs 14 and 16 of the stent graft 10 may include and be formed from at least one flexible layer of material such as PTFE, polymer meshes, composites of same or the like. For some embodiments, the main body portion 12, ipsilateral leg 14 and contralateral leg may include or be made from about 2 layers to about 15 layers or more of PTFE, polymer meshes, composites of the same or any other suitable material. The stent graft embodiment 10 is shown for purposes of illustration with an inflatable portion thereof in an inflated state with the inflatable portion full of a fill material 13.

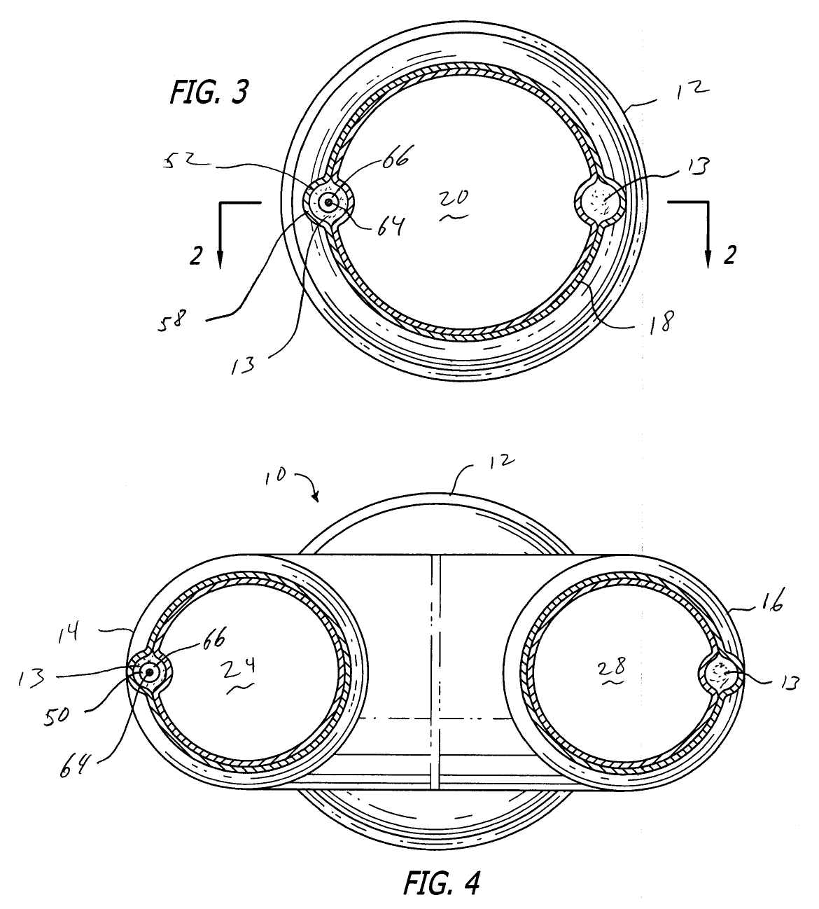

The main fluid flow lumen 20, shown in FIG. 3, of the main graft 12 generally may have a larger transverse dimension and area than a transverse dimension and area of either of the fluid flow lumens 24 or 28, shown in FIG. 4, and of the ipsilateral leg 14 or contralateral leg 16, respectively. A proximal anchor member or stent 30 is disposed at a proximal end 32 of the main graft 12 and may have a substantially cylindrical or tubular configuration for some embodiments. The proximal anchor member embodiment 30 shown in FIG. 1 includes a dual stent configuration including a first self-expanding stent member 34 disposed at a proximal position of the stent graft 10. The first self-expanding stent member 34 is formed from an elongate element having a generally serpentine shape with four crowns or apices at either end. Each distal apex or crown 36 of the first self-expanding stent member 34 is coupled to alternating proximal crowns or apices 38 of a second self-expanding stent member 40. The second self-expanding stent member 40 is disposed distally of the first self-expanding stent member 34 and is formed from an elongate element having a generally serpentine shape. A distal end 42 of the second self-expanding stent member 40 may be mechanically coupled to a connector ring 44 which is embedded in graft material of the proximal end 32 of the main graft 12, or directly coupled to perforations in a proximal edge region of the main graft 12.

Embodiments of the connector ring 44 may be generally circular or cylindrical in shape with regular undulations about the circumference that may be substantially sinusoidal or zig-zag in shape. Some embodiments of the first self-expanding stent member 34 may include outwardly extending barbs 46. Such barbs 46 may be integrally formed with the struts 48 of the self-expanding stent member 34, having sharp tissue penetrating tips that are configured to penetrate into tissue of an inside surface of a lumen within which the proximal stent 30 is deployed in an expanded state. Although the proximal anchor member 30 is shown as including first and second self-expanding stent members 34 and 40, the proximal anchor member 30 may include similar stents that are configured to be inelastically expanded with outward radial pressure as might be generated by the expansion of an expandable balloon from within either or both of the first and second stents. The connector ring 44 coupled to the second self-expanding stent member 40 may also be inelastically expandable for some embodiments. The self-expanding proximal anchor member embodiments 30, including each of the first and second self-expanding stent members 34 and 40, may be made from or include a superelastic alloy, such as NiTi alloy.

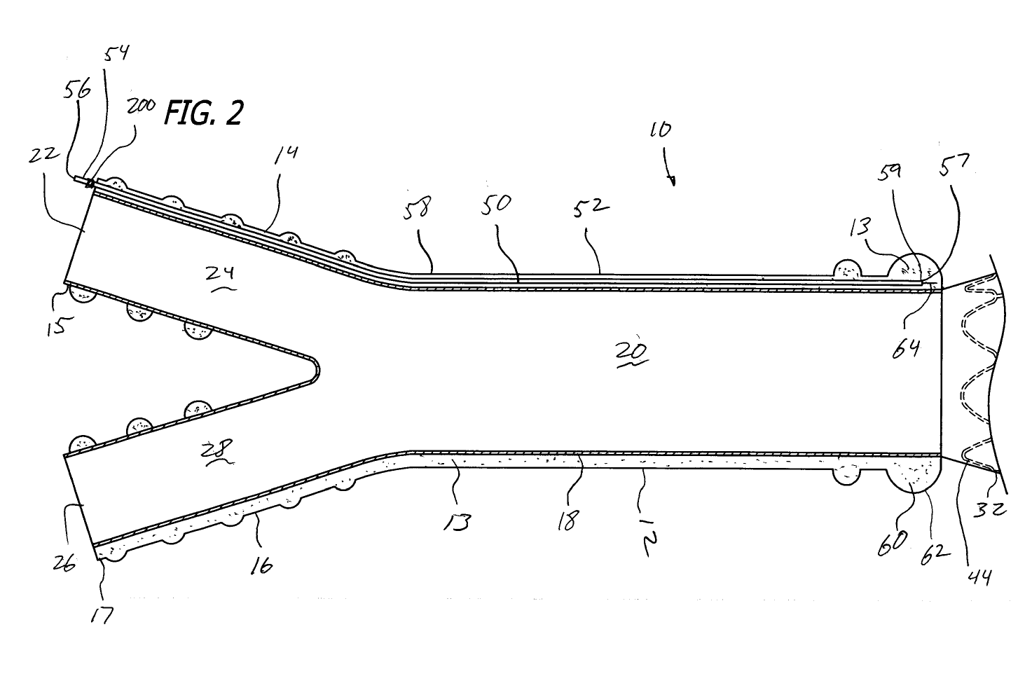

Some stent graft embodiments 10 may include an optional inflation conduit 50 which may serve as a fill manifold for inflation of an inflatable portion 52 of inflatable embodiments of stent grafts. Such inflation conduit embodiments 50 may be used to inflate inflatable portions 52 of the stent graft 10 from a desired site or sites within the inflatable portion 52. Referring to FIG. 2, the inflatable endovascular stent graft 10 includes at least one self-expanding stent member in the form of the proximal anchor member 30 and a flexible main graft body portion 12 secured to the anchor member 30. The graft portion includes the graft body portion 12 having a proximal end 32, ipsilateral and contralateral legs 14 and 16 having distal ends 15 and 17 respectively, and an inflatable portion 52. For some bifurcated stent graft embodiments, the distal ends of the legs may be considered the distal end of the graft body portion. The inflation conduit 50 is shown disposed within and extending into an interior volume of the inflatable portion 52. The inflation conduit 50 includes a distal end 54 with an inflation port 56 disposed at the distal end 54. The inflation port 56 is in fluid communication with an exterior portion of the graft body portion or may be otherwise disposed at a location or site that is exterior to an interior volume of the inflatable portion 52. The inflation port 56 is in fluid communication with an inner lumen 66 within the inflation conduit 50 which is in fluid communication with an outlet port 57. The outlet port embodiment 57 shown is disposed at a proximal end 59 of the inflation conduit 50. The inflation conduit 50 may extend from the distal end 15 of the ipsilateral leg 14 into an interior volume of the inflatable portion 52 of the stent graft 10.

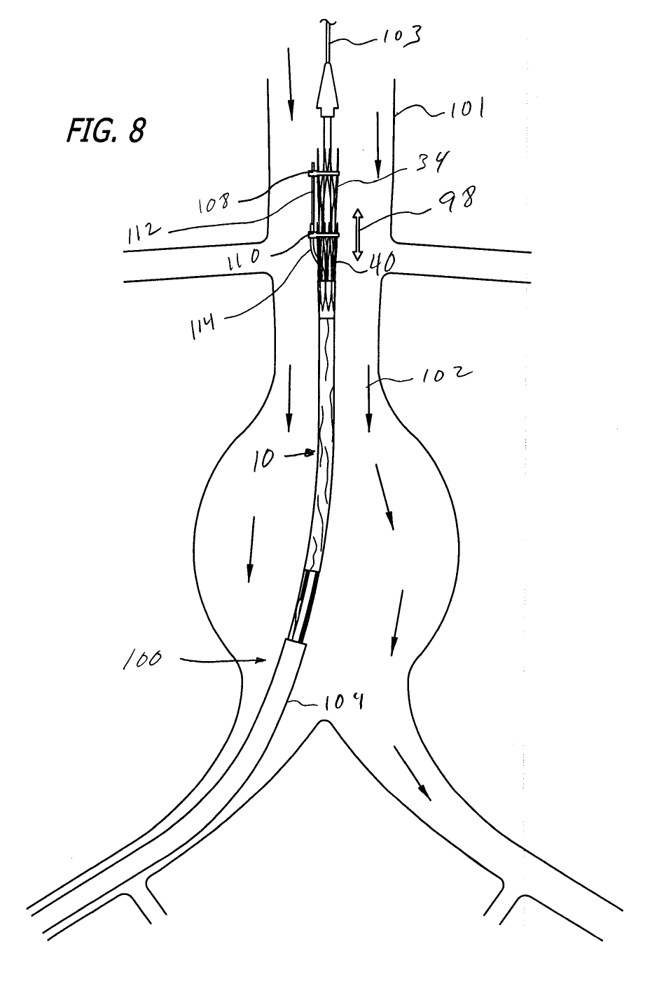

Some embodiments of the distal end 54 of the inflation conduit and inflation port 56 may be configured to releasably couple to a fill material conduit of a delivery system of the stent graft as shown in FIG. 8 and discussed below. For such embodiments, the fill material conduit of the delivery system may have a fill material lumen in fluid communication with the inflation port 56 and distal end 54 of the inflation conduit 50 so that fill material 13 can be injected into a proximal end of the fill material lumen of the delivery system. The pressurized fill material 13 then propagates through the fill material lumen and into the inflation port 56 of the inflation conduit 50. The pressurized fill material 13 then propagates through the inner lumen 66 of the inflation conduit to the outlet port or ports 57.

Inflation conduit embodiments 50 may include at least one outlet port 57 disposed at any desired position or desired positions within the inflatable portion 52 of the stent graft 10. The inflation conduit 50 has the single outlet port 57 positioned at a desired position within the inflatable portion and is configured to first fill the inflatable portion 52 from the desired position within an interior volume of the inflatable portion 52 of the stent graft 10. For some embodiments, the inflatable portion 52 of the stent graft 10 may include one or more inflatable channels formed from the flexible material of the graft body section including the main graft body section 12 and legs 14 and 16. The inflation conduit 50 of FIG. 2 is disposed within an interior volume of a longitudinal inflatable channel 58 of the network of inflatable channels and is configured to fill the network of inflatable channels from a desired position within an interior volume 60 of a proximal inflatable cuff 62 of the graft body portion of the stent graft 10.

For the particular stent graft and inflation conduit configuration shown in FIG. 2, the proximal inflatable cuff 60 may be filled first with fill material 13 after the proximal anchor member 30 has been deployed. That is, when fill material 13 is emitted under pressure from the outlet port 57 of the inflation conduit 50, the fill material will first begin to fill the proximal inflatable cuff 60. This arrangement may allow a seal to be formed between an outside surface of the proximal cuff 62 and a luminal surface of the patient's vasculature at the initial inflation stage. Such a seal may force a flow of blood through the main lumen 20 of the stent graft 10 and allow the stent graft main body 12 to open sequentially in a “windsock” type process. A windsock type deployment process may be useful in some circumstances in order to maintain control of the deployment process of the stent graft 10.

The inflation conduit 50, an inner lumen 66 of which is in communication between a location outside the inflatable portion 52 of the stent graft 10 and an interior volume of the inflatable portion 52, may be disposed within any desired portion of the inflatable portion 52. Inflation conduit embodiments 50 disposed within the interior volume of the inflatable portion 52 may include a variety of configurations with regard to the size or area and position of the outlet port or ports 57. The inflation conduit 50 shown in FIG. 2 has a single outlet port 57 disposed at the proximal end 59 of the inflation conduit 50. The outlet port 57 is disposed within the interior volume 60 of the proximal inflatable cuff 62 disposed at the proximal end 32 of the graft body portion. The position of the outlet port 57 is configured to emit fill material injected into the inflation conduit 50 from the outlet port 57 so as to first inflate the proximal inflatable cuff 62 as discussed above. The inflation conduit 50 extends distally of the outlet port 57 and is disposed within the longitudinal inflatable channel 58 of the inflatable portion of the stent graft 10 for the embodiment shown. The longitudinal inflatable channel 58 extends distally from the proximal inflatable cuff 62.

Some inflation conduit embodiments 50 may be made from a flexible, collapsible material, such as PTFE. For such embodiments, it may be desirable to have an elongate bead 64 disposed within an inner lumen 66 of the inflation conduit 50. Such a bead 64 may be made from a flexible but substantially incompressible material, such as a solid PTFE extrusion with or without a radiopaque additive doping (bismuth, barium or other commonly used radiopaque extrusion additives). Bead embodiments may be useful for maintaining a patent lumen passage 66 through the inflation conduit 50 when the stent graft 10 and inflatable portion 52 thereof is in a constrained state prior to deployment. This configuration may also allow the inflation conduit 50 of the stent graft 10 to be visible under fluoroscopy for orientation purposes throughout the deployment process prior to inflation of the inflatable portion with fill material. For the embodiment shown, the bead 64 extends distally from a position just proximal the outlet port 57 at the proximal end 59 of the inflation conduit 50. A distal end of the bead 64 may be secured at any axial position within the inner lumen 66 of the inflation conduit 50, but may also be secured to a distal portion of the inflation conduit 50 as discussed in more detail below.

The inflation conduit embodiment 50′ shown in FIGS. 5-5B includes a plurality of outlet ports 57′ along a wall of the inflation conduit 50′ and at a distal end 54′ of the inflation conduit 50′. The inflation conduit embodiment 50′ also includes an inflation port 56′ which may be in fluid communication with an exterior portion of the graft body portion. The inflation conduit embodiment 50′ may extend from the distal end of the graft body portion and into an interior volume of the inflatable portion 52 as discussed above with regard to inflation conduit embodiment 50. The plurality of outlet ports 57′ may be in fluid communication with an interior volume of the inflatable longitudinal channel 58 and proximal cuff 62 of the graft body portion.

The plurality of outlet ports 57′ may also be configured in size and location so as to inflate the inflatable portion 52 substantially evenly with respect to a longitudinal axis of the graft body portion or inflatable channel 58. For example, the outlet ports 57′ may be disposed coextensively with the length of the interior volume of the longitudinal inflatable channel 58 and each outlet port 57′ have a progressively larger area in a direction from a distal end of the inflation conduit 50′ to a proximal end 59′ of the inflation conduit 50′ as shown in FIG. 5. Such a configuration may be arranged to accommodate a differential pressure gradient of fill material within the inflation conduit lumen 66′ from the distal end 54′ of the inflation conduit 50′ to the proximal end 59′ of the inflation conduit and thereby allow a substantially even fill of the inflatable portion of the stent graft 10. A bead 64 is shown disposed within the inner lumen 66′ of the inflation conduit embodiment 50′ of FIGS. 5-5B. The elongate bead 64 has a substantially round transverse cross section which is sized to be smaller than a transverse dimension of the inner lumen 66′ of the inflation conduit 50′ when in an expanded state. The bead 64 extends distally from a position proximal of the proximal end 59′ through the inner lumen 66′ to a junction 74′ between tubular sections of the inflation conduit 50′. The joint between the distal end of the bead 64 and the inflation conduit components of inflation conduit 50′ may be accomplished by any of the methods discussed herein.

FIGS. 6A-6F illustrate the inflation conduit embodiment 50 discussed above in greater detail. The inflation conduit has a single outlet port 57 disposed at a proximal end 59 of the inflation conduit 50 and bead 64 disposed within the inner lumen 66 of the inflation conduit 50. The figures also show construction detail embodiments of the inflation conduit embodiment 50 including the attachment of the distal end 68 of the bead 64 to the inflation conduit components. FIG. 6B shows a top view and a side view of the distal end 68 of the bead 64. The distal end 68 shown has a flattened configuration suitable for bonding of the distal end 68 to the inflation conduit component structure. The nominally round cross section of the bead 64 is shown in FIG. 6C and the cross section of the flattened distal end 68 of the bead 64 is shown in FIG. 6D.

FIG. 6E shows a junction between a distal tubular member 70 and proximal tubular member 72 of the inflation conduit embodiment 50 of FIG. 6A. The flattened distal end 68 of the bead 64 is secured in the junction 74 between the distal and proximal tubular members 70 and 72 with the bead 64 extending proximally from the joint 74. The flattened distal end 68 of the bead 64 may be secured to the surfaces of the tubular member by any suitable method or material. Bonding methods or materials may be used (not shown) between the junction 74 of the distal and proximal tubular members 70 and 72 of the inflation conduit 50 including adhesive bonding, thermal bonding or welding, solvent bonding or the like. For such embodiments, the surfaces of the flattened distal end 68 of the bead 64 may be secured to an inside surface of the proximal tubular member 72 of the inflation conduit 50 and outer surface of the distal tubular member 70 of the inflation conduit at the junction 74 therebetween. The flattened configuration of the distal end 68 of the bead 64 may be useful in maintaining the continuity of the junction 74 between the tubular members 70 and 72 and a fluid tight seal therebetween. For some embodiments, the amount of axial overlap at the junction between the distal tubular member 70 and proximal tubular member 72 may be about 2 mm to about 20 mm.

As shown in FIG. 6F, the flattened distal end 68 of the bead 64 may also be so secured to an inside surface of either the proximal tubular member 72 or distal tubular member 70 for some embodiments. In general, the flattened distal end 68 of the bead 64 may be secured to an inside luminal surface of the inflation conduit 50 without being disposed between the distal tubular member 70 and proximal tubular member 72 of the joint 74. The flattened distal end 68 may be secured with any of the bonding materials or methods discussed above with regard to the embodiment shown in FIG. 6E. The bead 64 may extend from the flattened distal end 68 proximally through the inner lumen 66 of the inflation conduit 50. For some embodiments, the bead may extend through the entire lumen 66 to the proximal end 59 of the inflation conduit 50 or it may terminate at any desired position within the inner lumen 66 of the inflation conduit 50. The flattened distal end 68 of the bead 64 may also optionally be secured to an outside surface of the proximal tubular member 72 with the bead 64 extending proximally towards the proximal end of the inflation conduit 50. Such a bead configuration may be disposed adjacent an outer surface of the proximal tubular member 72 of the inflation conduit 50.

The distal tubular member 70 may include a tubular material that has sufficient rigidity in order to maintain the inner lumen 66 when the stent graft 10 is in a constrained state and connected to a fill lumen within a delivery catheter as shown in FIG. 8 and discussed below. Suitable materials for the distal tubular member 70 of the inflation conduit 50 may include PTFE. As discussed above, the proximal tubular member 72 of the inflation conduit 50 disposed within the interior volume of the inflatable portion 52 of the stent graft 10 may be made from a substantially flexible material that is collapsible. Such an arrangement may provide a distal portion of the inflation conduit 50 that has sufficient structural rigidity for effective coupling to a removable fill material tube or lumen of a delivery system. Such effective releasable coupling may be made while the proximal portion 59 of the inflation conduit 50 disposed within the inflatable portion 52 of the stent graft 10 may be flexible for maintaining the flexibility of the stent graft 10 overall. An outer surface of the inflation conduit 50 is typically sealed to the flexible material of the inflatable portion 52 of the stent graft 10 at or near the junction 74 between the distal and proximal tubular members 70 and 72 of the inflation conduit 50. As such, the interior volume of the inflatable portion 52 of the stent graft 10 may be sealed and fluid tight except for the inner lumen 66 of the inflation conduit 50 which provides a passageway from a position exterior to the inflatable portion 52 into the interior volume of the inflatable portion 52.

For some embodiments, the inflation conduit 50 may have an overall length of about 90 mm to about 135 mm, an outer transverse dimension of about 1.2 mm to about 2.2 mm, and a transverse dimension of the inner lumen 66 of about 1 mm to about 1.8 mm. The wall thickness of the inflation conduit 50 may be about 0.05 mm to about 0.1 mm for some embodiments. The length of the inflation conduit 50 may include a length of about 35 mm to about 85 mm for the distal tubular member 70 and a length of about 50 mm to about 55 mm for the proximal tubular member 72. For some embodiments, the outlet ports 57 in the wall of the inflation conduit 50 may have a transverse dimension of about 0.1 mm to about 1.3 mm. For some embodiments, the bead 64 may have a length of about 55 mm to about 270 mm and an outer transverse dimension of about 0.25 mm to about 0.5 mm.

As discussed above, FIGS. 7-20A illustrate positioning and deployment method embodiments of a bifurcated inflatable stent graft, such as the bifurcated inflatable stent graft embodiment 10, shown in FIGS. 1-4. For some embodiments, a method of deploying an inflatable endovascular stent graft 10 includes advancing a delivery catheter that includes the endovascular stent graft 10 in a radially constrained state to a deployment site within a patient's vasculature. Once at a desired treatment site within the patient's vasculature, the endovascular stent graft 10 may be partially deployed so as to allow at least a portion of a proximal self-expanding member of the endovascular stent graft 10 to radially expand. An imaging system may be aligned relative to the patient's body such that an imaging axis of the imaging system is substantially orthogonal to a longitudinal axis of a tubular main body portion of the endovascular stent graft 10. The partially deployed endovascular graft 10 may then be positioned in an axial direction to a desired position within the patient's vasculature to assure treatment of the treatment site and to assure proper location of the graft body and proximal anchor member with regard to the anatomy of the patient's vasculature. The proximal self-expanding member of the endovascular graft 10 may then be fully deployed so as to engage an interior luminal surface within the patient's vasculature.

Referring to FIG. 7, a delivery catheter 100 containing a stent graft 10 in a radially constrained state is advanced to a deployment site within a patient's vasculature 101. The delivery catheter 100 may be advanced over a guidewire 103 such that a proximal end of the stent graft 10 is disposed towards a flow of blood, as indicated by arrow 102, within the patient's vasculature 101. The constrained stent graft 10, which is disposed beneath an outer sheath 104 of the delivery catheter 100, may be axially positioned within the patient's vasculature (as indicated by arrow 98) adjacent a treatment site. The treatment site shown includes an abdominal aortic aneurysm 106 within a patient's vasculature 101. Such a delivery catheter 100 may include some or all of the features, dimensions or materials of delivery systems discussed in commonly owned U.S. Patent Application Publication No. 2004/0138734, published Jul. 15, 2004, filed Oct. 16, 2003, by Chobotov et al., titled “Delivery System and Method for Bifurcated Graft” which is incorporated by reference herein in its entirety.

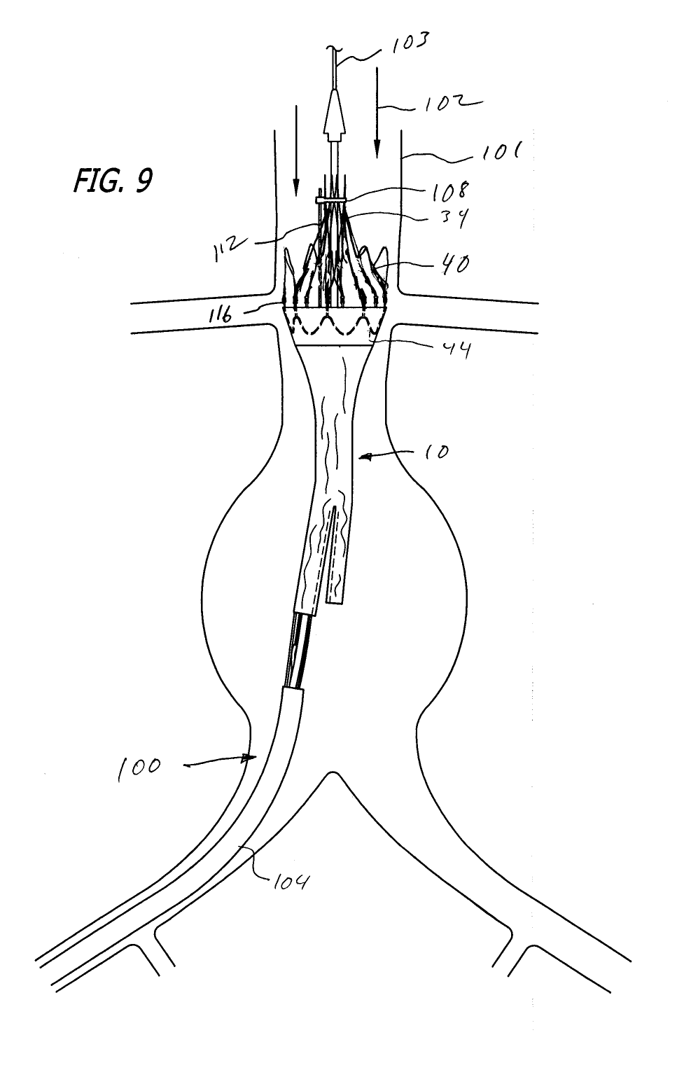

Once the delivery catheter 100 has been disposed at a desired treatment site, the outer sheath 104 of the delivery catheter 100 may be retracted distally as shown in FIG. 8. As shown in FIG. 8, once the outer sheath 104 of the delivery catheter 100 is retracted, the stent graft 10 which is releasably secured to the delivery catheter 100 with the proximal anchor member 30 in a constrained state is exposed. For some embodiments, retraction of the outer sheath 104 from the stent graft 10 may put the stent graft 10 in a partially deployed state. At this stage, the proximal anchor member 30 of the stent graft 10 is still restrained by first belt member 108 and second belt member 110 disposed about the first self-expanding stent member 34 and second self-expanding stent member 40 of the proximal anchor member 30 respectively. Looped ends of the first belt member 108 may be releasably secured together with a first release wire 112 which passes through the looped ends of the first belt member 108. Looped ends of the second belt member 110 may be releasably secured together with a second release wire 114 which passes through the looped ends of the second belt member 110. The distal or second belt member 110 may be released by retraction in a distal direction of the second release wire 114 so as to remove the circumferential constraint of the second belt member 110 about the second stent member 40 of the proximal anchor member 30. Removal of the circumferential constraint of the second belt member 110 may be used to partially deploy the stent graft 10 as illustrated in FIG. 9. In FIG. 9, the second self-expanding member 40 has radially expanded while the first self-expanding member 34 remains in a constrained state radially and circumferentially constrained by the first belt member 108. At this stage, finalizing the axial position of the stent graft 10 relative to the anatomy of the patient's vasculature 101 may be accomplished with the use of some radiopaque marker devices 116 that facilitate alignment of an imaging system with the stent graft 10 and the patient's anatomy or vasculature 101.

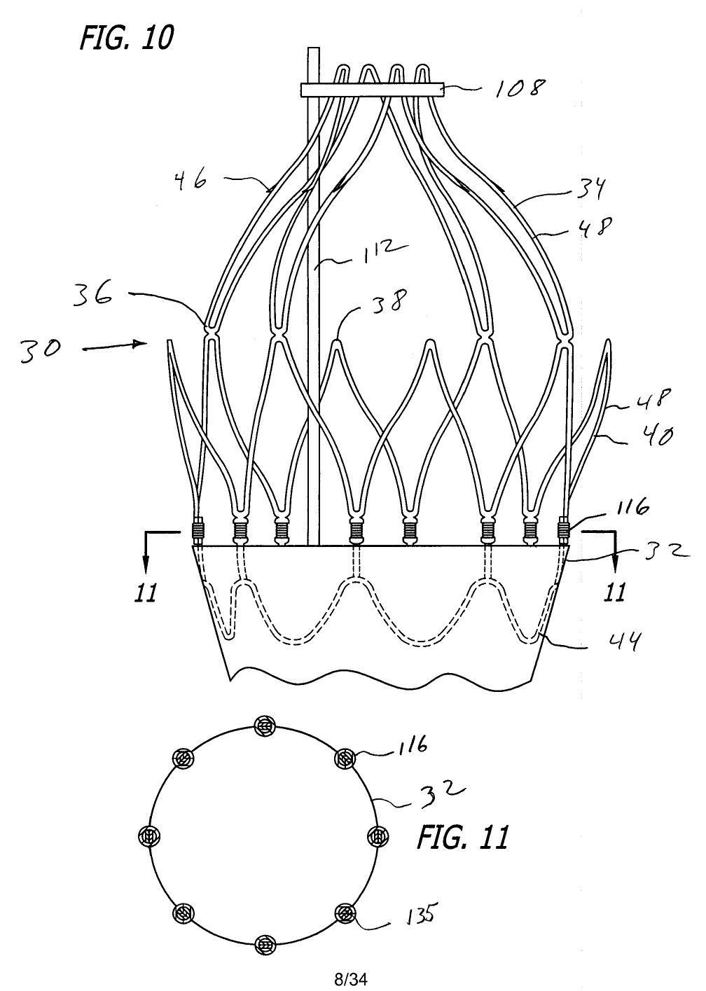

FIGS. 10 and 11 illustrate in an enlarged view of a portion of the stent graft 10 with portions of the delivery catheter 100 not shown for clarity of illustration. FIGS. 10 and 11 show an arrangement of some radiopaque markers 116 of the stent graft 10 that may be used to facilitate alignment of the stent graft 10 and alignment of an imaging system with respect to the stent graft 10. As discussed above, the stent graft 10 includes a tubular flexible main body portion 12, a proximal self-expanding stent member 30 and a plurality of radiopaque markers 116 circumferentially disposed about a tubular portion of the endovascular stent graft 10. The radiopaque markers 116 shown in FIGS. 10 and 11 lie in a circular pattern in a plane that is substantially orthogonal to a longitudinal axis 118 of the tubular main body portion 12 of the stent graft. More particularly, for the embodiment shown, the plurality of radiopaque markers 116 is evenly spaced and disposed about a circumference of a distal end of the second self-expanding stent member 40 of the stent graft 10. Even more particularly, the plurality of radiopaque markers 116 include helically wound wire members which are disposed about connector members 120 that mechanically couple the second self-expanding stent member 40 of the proximal anchor member 30 to the connector ring 44 disposed within the proximal end 32 of the main body portion 12 of the stent graft 10.

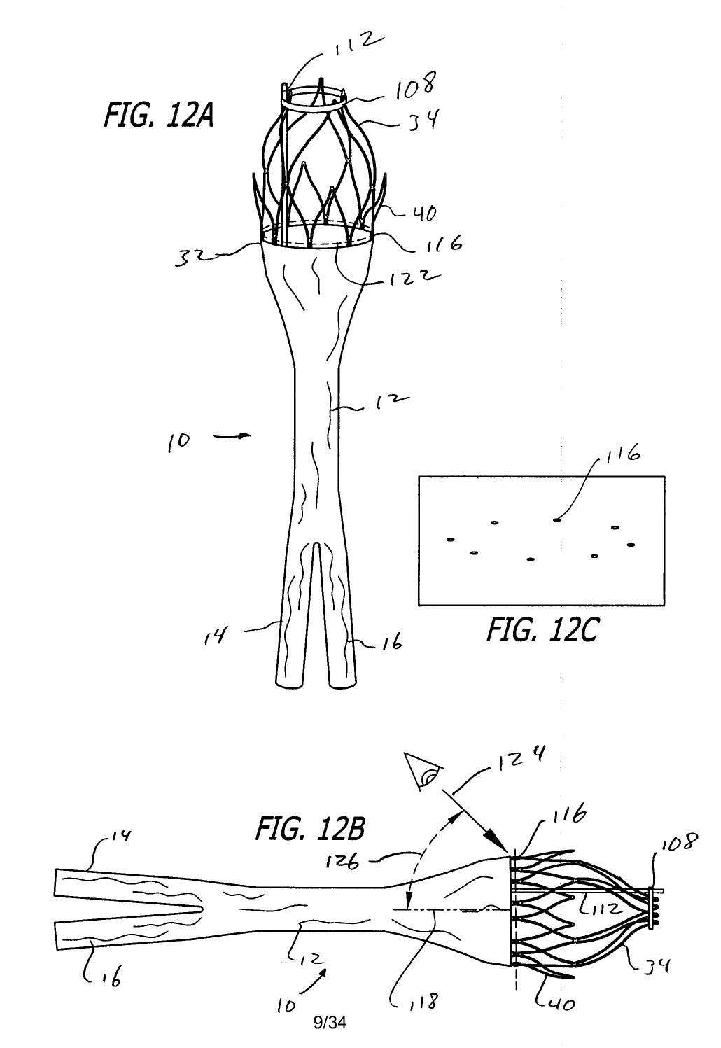

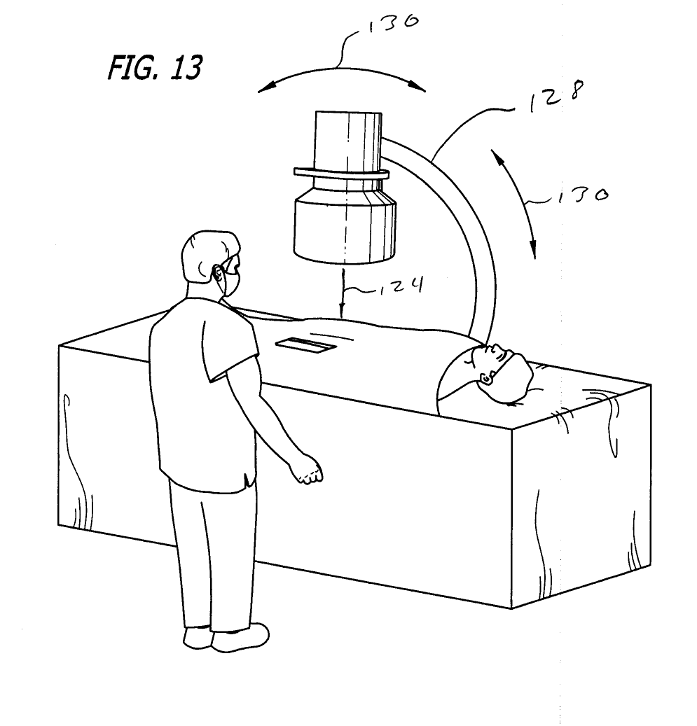

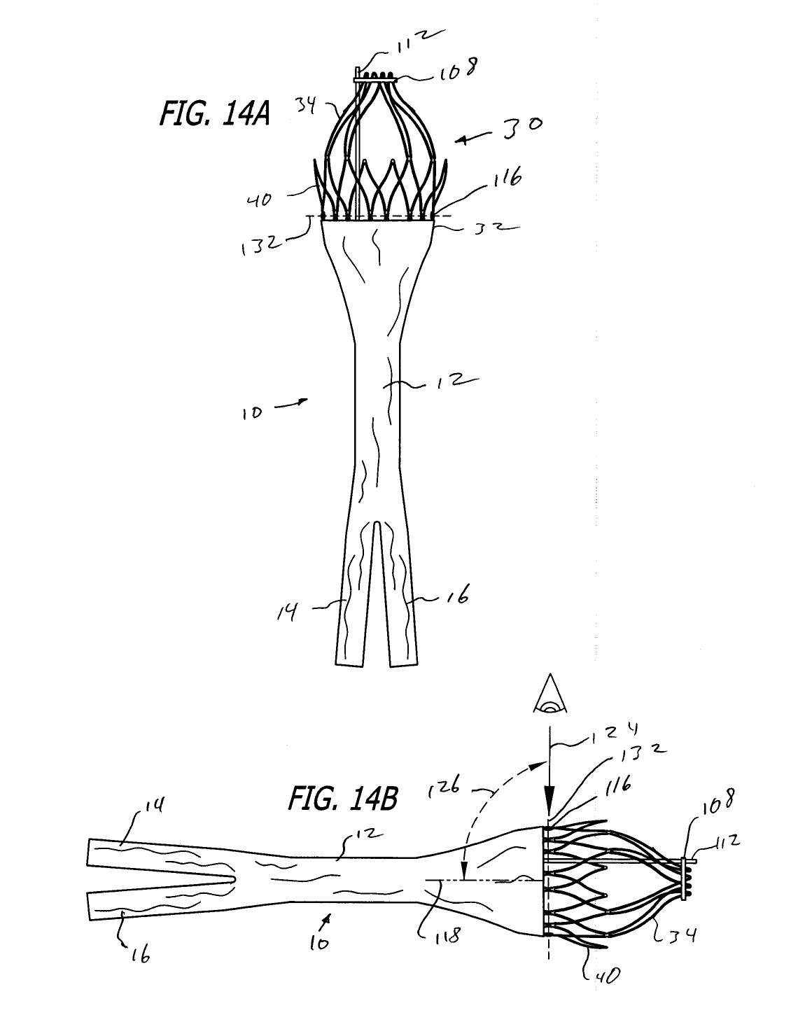

As can be seen in FIG. 11, the radiopaque markers 116 are disposed in a substantially circular evenly spaced arrangement about a perimeter of the stent graft 10 which is in a partially deployed state. This arrangement of radiopaque markers 116 allows an observer of the stent graft 10 during deployment within the patient's body to achieve a view of the stent graft 10 which is substantially orthogonal to the longitudinal axis 118 of the stent graft 10 by aligning the radiopaque markers 116 into a linear configuration. When observed via an x-ray type imaging system from a non-orthogonal view, the radiopaque markers 116 appear as an ellipse as indicated by the dashed line 122 shown in FIG. 12A. This non-orthogonal view is further illustrated by the observer line of sight 124 relative to the longitudinal axis 118 of the stent graft main body portion 12 shown in FIG. 12B. The angle of the line of sight 124 relative to the longitudinal axis 118 of the stent graft main body portion 12 is indicated by the arrow 126. For an orthogonal view, the angle indicated by arrow 126 would be about 90 degrees. FIG. 12C illustrates an image of the radiopaque markers 116 that might be viewed by an observer from a non-orthogonal line of sight using an imaging system that registers an image only of the markers and not the remainder of the stent graft structure, such as an x-ray type or fluoroscopy type imaging system 128 as shown in FIG. 13.

In order to achieve a substantially orthogonal view angle, the imaging system 128 used to image the stent graft 10 and patient's vasculature 101 may be adjusted in a variety of translational and angular axes 130 relative to the patient, as shown in FIG. 13. Such angular and translational adjustment may be made until a substantially orthogonal view angle is achieved as shown in FIGS. 14A and 14B wherein the image of the plurality of radiopaque markers 116 is aligned linearly to the observer, as indicated by dashed line 132. As shown in these figures, the imaging system 128 is aligned relative to the patient's body by aligning the plane defined by the plurality of radiopaque markers 116 substantially along the imaging axis or line of sight 124 of the imaging system 128 or view angle of an observer. In such an arrangement, the radiopaque markers 116 are disposed about a circumference of a tubular portion of the stent graft 10 and lie in a plane which is substantially orthogonal to a longitudinal axis 118 of the tubular main body portion 12 of the endovascular stent graft 10. Although an observer's eye is schematically illustrated in FIGS. 12B and 14B, the perspective illustrating the view angle 126 may be indicative of either direct observation, a view angle or imaging axis 124 of an imaging system 128 such as shown in FIG. 13, or the view angle or imaging axis 124 of any other suitable imaging system. Other features of the stent graft 10 visible under fluoroscopy or other suitable forms of imaging which are suitably oriented about the device may be used instead of, or in conjunction with, the radiopaque markers 116 to facilitate orthogonal orientation of the imaging axis or view 124. For example, other suitable features of the stent graft 10 may include the self-expanding members 34 and 40, or components thereof, stent connector members 135 (as shown in FIG. 11) or any other suitable structure, such as structures that are symmetrically disposed about the longitudinal axis of the main graft portion 12.

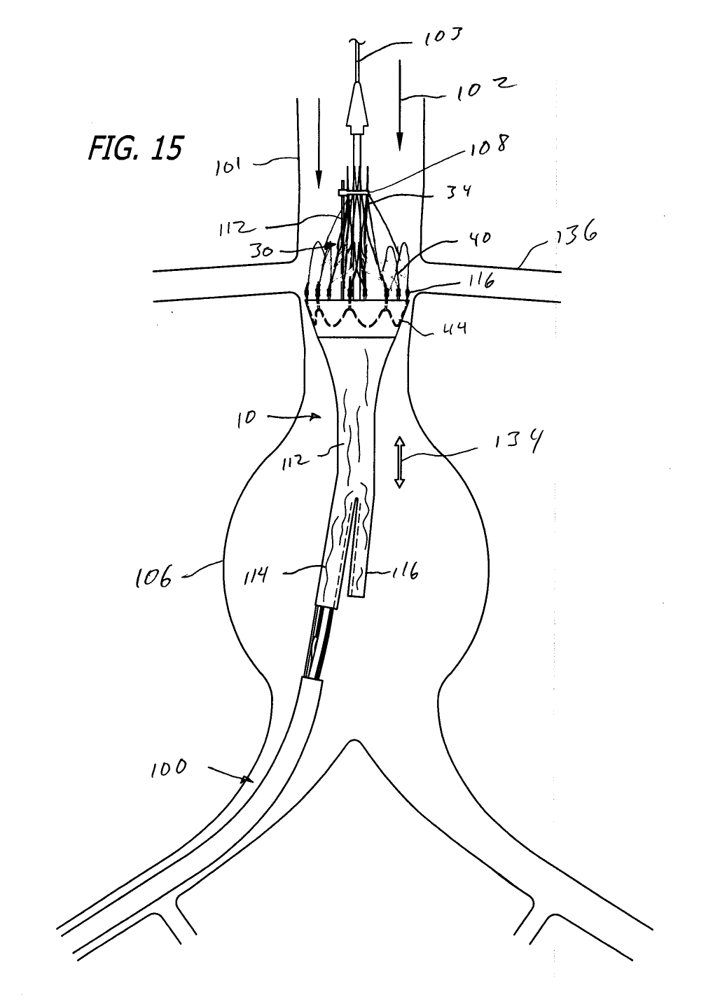

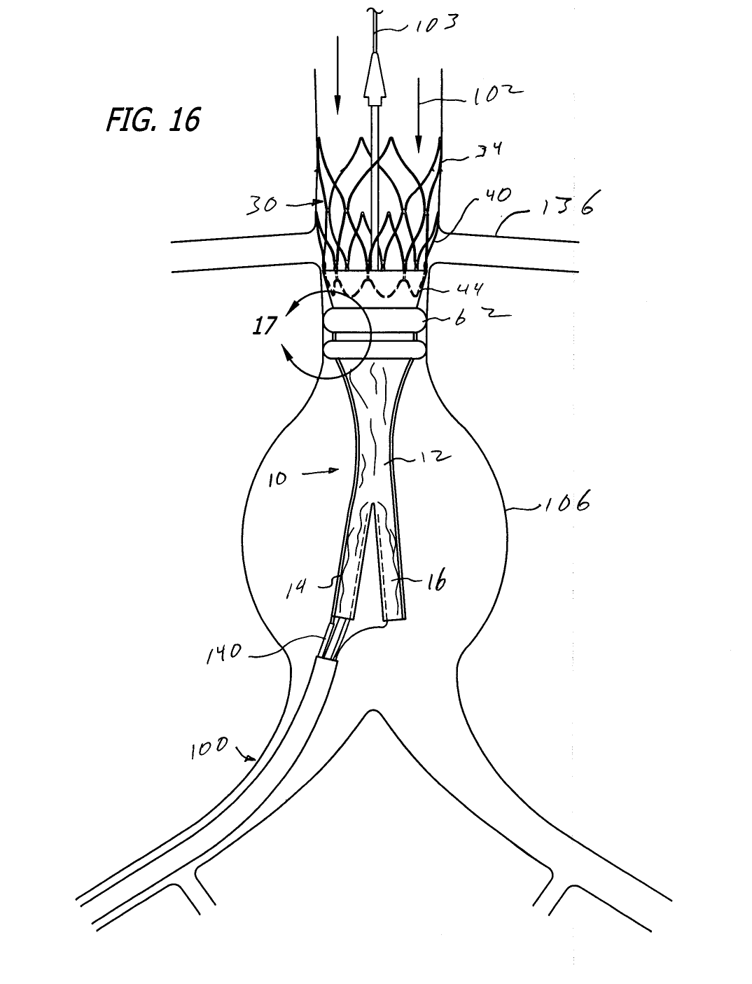

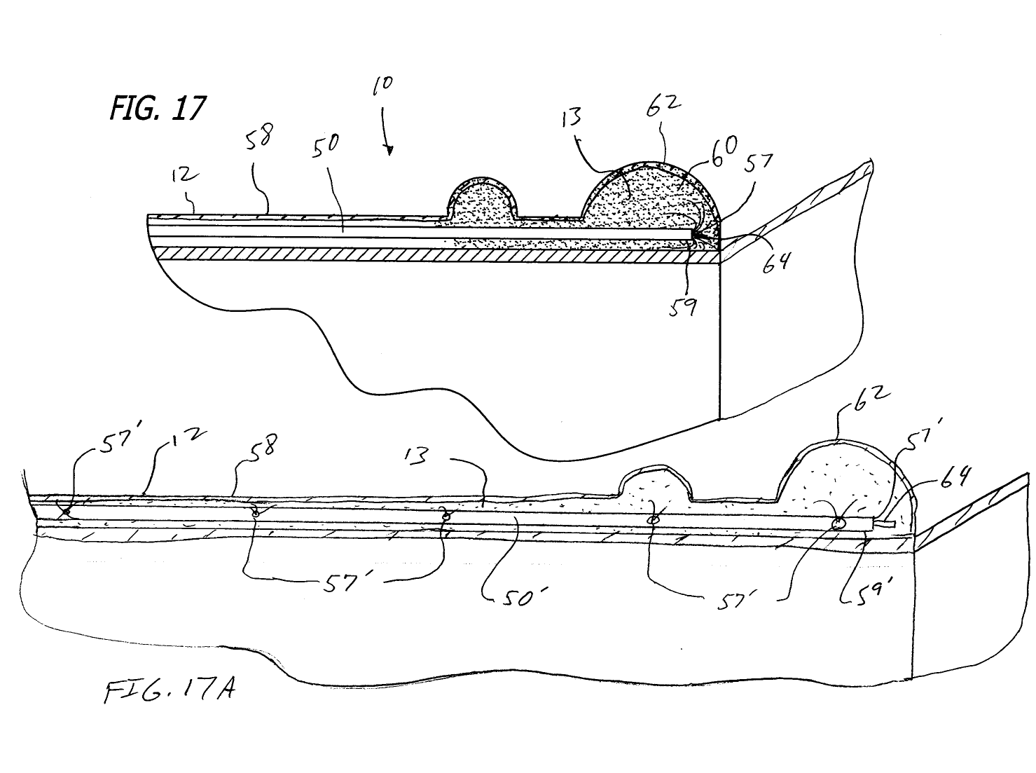

Once a substantially orthogonal view angle is achieved, an accurate axial position of the partially deployed stent graft 10 relative to the patient's vasculature 101 may be achieved, avoiding parallax, ensuring precise placement of the stent graft 10 relative to significant branch vessels 136 or other anatomic reference points. Parallax in some circumstances can cause error in axial placement of the stent graft 10 relative to the intended target site. Accurate positioning may be achieved with axial movement and adjustment of the stent graft by manual manipulation of a proximal portion of the delivery catheter 100 as indicated by arrow in FIG. 15. As shown in FIG. 15, the stent graft 10 is positioned such that the proximal end 32 of the main body portion 12 of the stent graft 10 is aligned distal of the ostium of the renal arteries 136. Once the stent graft 10 in the partially radially constrained state is axially aligned, the proximal anchor member 30 may then be fully deployed so as to engage and be secured to the luminal wall or interior luminal surface 138 of the patient's vasculature 101 as shown in FIG. 16. Once the proximal anchor member 30 is fully deployed, the inflatable portion 52 of the stent graft 10 including the network of inflatable channels may be inflated with a fill material 13. For some embodiments, the network of inflatable channels may be filled from a desired site within the inflatable portion 52. More specifically, the inflatable portion 52 may be inflated with fill material 13 from a proximal end or portion thereof as shown in more detail in FIG. 17.

FIG. 17 illustrates a proximal portion of the network of inflatable channels or inflatable portion 52 in longitudinal section being inflated with fill material 13. The fill material 13 is being dispensed or otherwise ejected under pressure from a proximal outlet port 57 of the inflation conduit 50 disposed within a proximal portion of the inflatable portion 52 of the stent graft 10. As shown, the proximal inflatable cuff 62 is full of fill material 13 and the boundary of fill material is extending distally along the longitudinal inflatable channel 58. For some embodiments, inflating a proximal portion of the inflatable portion 52 includes inflating the proximal cuff 62. In some instances, the inflation conduit 50 or portions thereof may become compressed with a corresponding compression and restriction of the inner lumen 66 of the inflation conduit 50 when the stent graft 10 is in a radially constrained state, such as when loaded on a delivery catheter 100. For some embodiments, the inner surface of the inner lumen 66 of the inflation conduit 50 may temporarily adhere or stick to itself which may hinder the passage of inflation or fill material 13 therethrough. In circumstances such as this, it may be useful to maintain an inner lumen opening within the inflation conduit 50 prior to the inflation process with an embodiment of the bead 64 disposed within the lumen 66 of the inflation conduit 50 as shown. As discussed above, the bead 64 may be useful for maintaining at least a small luminal opening within the inner lumen 66 of the inflation conduit 50, even when the inflation conduit 50 is in a compressed or collapsed state. Once fill material 13 begins to flow through the small luminal opening created between an outer surface of the bead 64 and the adjacent inner luminal surface of the inflation conduit 50, the force of the pressure of the fill material 13 will typically overcome the adherence of the luminal wall surface to itself and fully open the inner lumen 66 of the inflation conduit 50.

FIG. 17A illustrates an inflatable portion of the stent graft being inflated by an inflation conduit, such as the inflation conduit 50′ illustrated in FIGS. 5-5B. The inflation conduit 50′ shown has a plurality of outlet ports 57′ disposed along a wall portion of the inflation conduit 50′. This configuration allows the fill material 13 to be emitted from the inflation conduit 50′ at any desired location along the inflation conduit lumen 66′. Further, the amount of fill material 13 emitted from various portions of the inflation conduit 50′ may be determined by the size and density of outlet ports 57′ at any given position on the inflation conduit 50′. For the embodiment shown in FIG. 17A, each successive outlet port 57′ in a proximal direction is larger in area or transverse dimension than the outlet port 57′ located distally thereof. Such an arrangement may be configured to substantially counter the pressure gradient between the distal end 54′ of the inflation conduit 50′ and the proximal end 59′ of the inflation conduit 50′. The inflatable channels 58 and cuffs 62 of the inflatable portion 52 of the stent graft 10 may thereby be inflated evenly along the length of the stent graft 10. Any other suitable or desirable outlet port design may also be used in order to inflate the inflatable portion 52 of the stent graft 10 as desired. Although inflation conduits extending substantially the full length of the inflatable portion 52 have been illustrated, inflation conduits having a single outlet port at a proximal end thereof or multiple outlet ports disposed along a length thereof, may have a length that terminates at a different position within the inflatable portion 52. For some embodiments, the inflation conduit 50′ may terminate at a position at about half the axial length of the longitudinal inflatable channel 58.

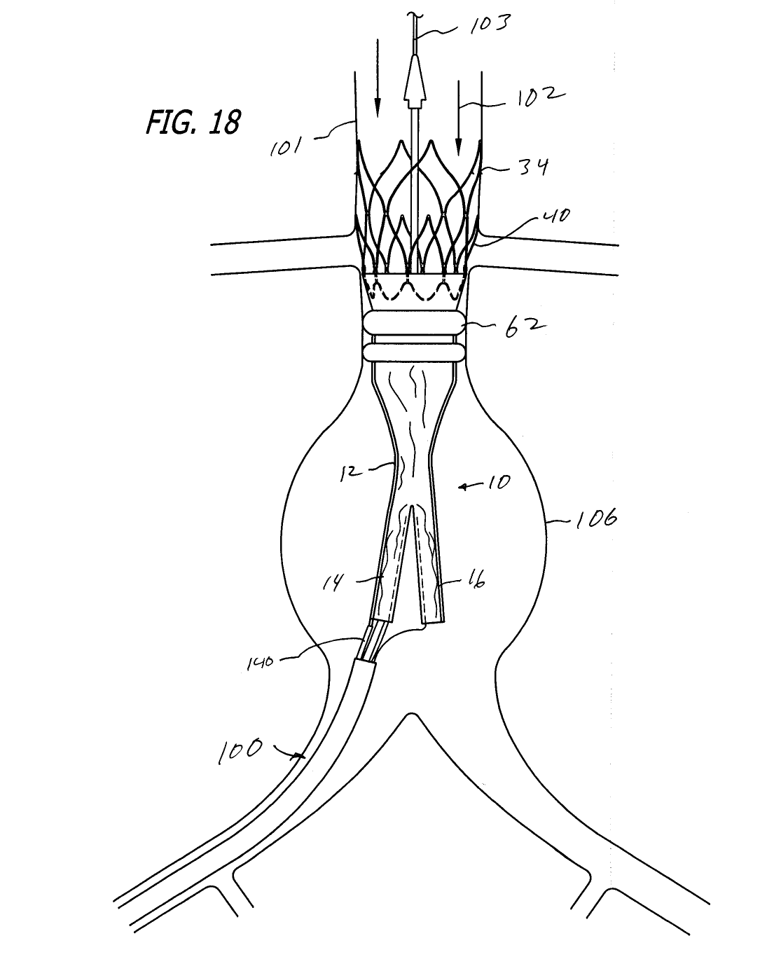

Upon injection of fill material 13, the proximal inflatable cuff 62 may be inflated and expanded, as shown in FIG. 17, so as to form a seal with the luminal surface of the patient's aorta 101 or other vascular passageway, as shown in FIG. 18. For some embodiments, the proximal cuff 62 of the stent graft 10 may be inflated and form a seal with a luminal surface of the patient's vasculature 101 before the inflatable portion 52 is completely filled. Once a seal is formed between an outside surface of the stent graft 10, more particularly, an outside surface of the proximal inflatable cuff 62 of the stent graft 10, and the inner luminal surface of the aorta 101, blood may begin to flow forcefully through the main lumen or conduit 20 of the stent graft 10 as shown in FIG. 18. Such a flow of blood through the main lumen 20 of the main body portion 12 may produce a windsock type action that sequentially opens the main lumen 20 in a distal direction due to the blood flow.

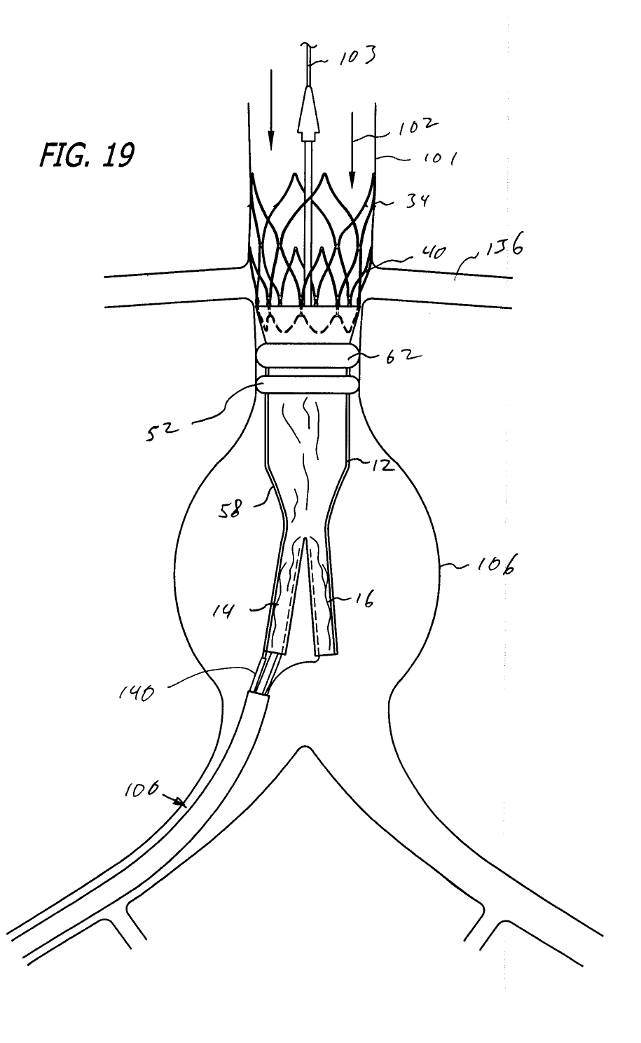

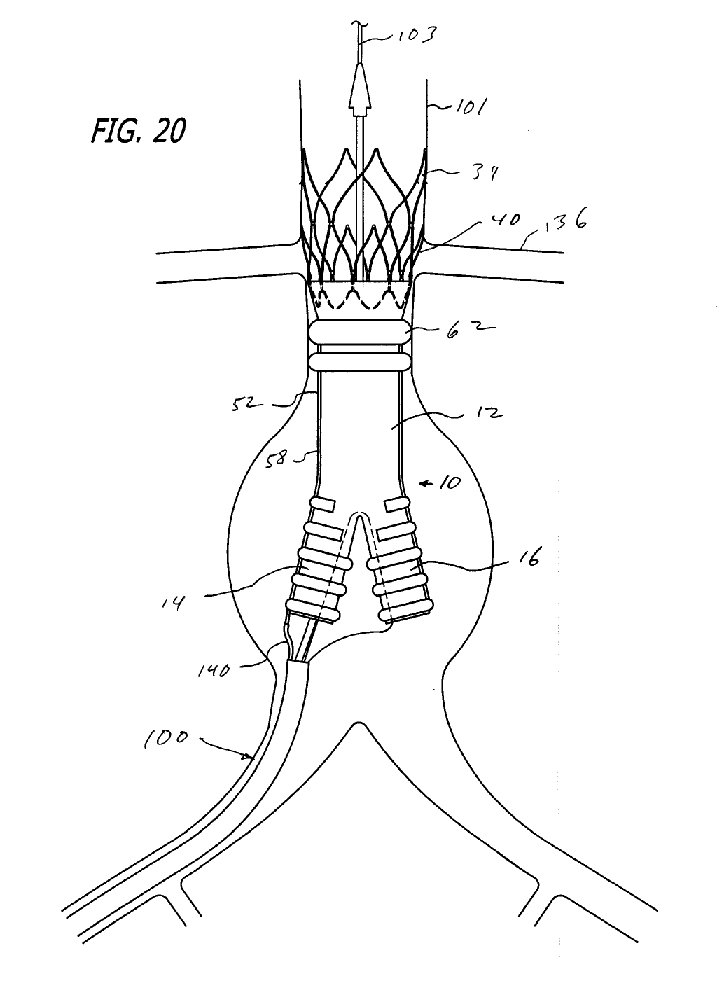

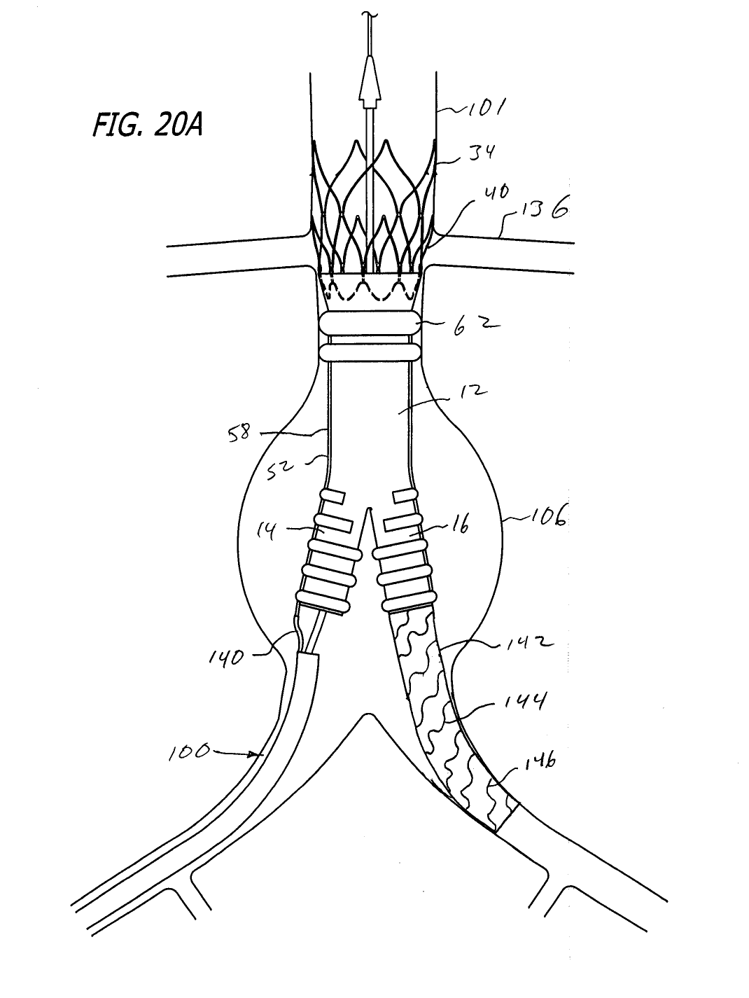

FIG. 18 illustrates the initiation of such a windsock process whereby the blood flow or pressure within the passage or lumen 20 of the main body portion 12 opens the lumen to a full or substantially full transverse dimension or diameter. FIG. 19 illustrates the main body portion 12 of the stent graft 10 more fully filled by blood flow therethrough. The windsock filling process may typically continue until the entire main body portion 12 and leg portions 14 and 16 are filled or substantially filled and expanded to their full transverse dimension. As the possible windsock process is taking place, or thereafter, fill material 13 may continue to be dispensed from the inflation conduit 50 until the inflatable portion 52 of the stent graft 10 is completely filled as shown in FIG. 20. FIG. 20 illustrates the stent graft 10 with the proximal anchor member 30 completely deployed and engaged with the inner luminal wall of the patient's vasculature 101, with the inner lumen 20 of the main body portion 12 and legs 14 and 16 of the stent graft 10 fully opened and with the inflatable portion 52 of the stent graft 10 completely inflated. The complete inflation of the inflatable channels 58 and cuffs 62 of the stent graft 10 may produce a structure that is sealed well against the inner luminal surface of the patient's vasculature 101, conforms to the patient's vasculature 101, and is sufficiently rigid to maintain a stable structure. The completely inflated structure also maintains luminal passages for blood flow and is sufficiently flexible to maintain conformance to the patient's vasculature during pulsatile cardiac cycling and blood flow. Once the stent graft 10 has been fully deployed as depicted in FIG. 20, the fill tube 140 of the delivery catheter 100 coupled in fluid communication with the distal end 54 of the inflation conduit 50 may be uncoupled from the inflation conduit 50. In addition, the guidewire 103, fill tube 140 and delivery catheter structure generally may then be distally retracted from the deployed stent graft 10 and the patient's vasculature 101.

For the bifurcated embodiment in FIG. 20, once the main stent graft device 10 is deployed, additional leg extensions 142 may be deployed within the legs 14 and 16 of the stent graft 10 as shown in FIG. 20A. For some embodiments, an ipsilateral leg extension (not shown) and a contralateral leg extension 142 may be so deployed. For example, an ipsilateral stent graft extension may be deployed into the ipsilateral leg 14 of the bifurcated stent graft 10 and a contralateral stent graft extension 142 may be deployed into the contralateral leg 16 of the bifurcated stent graft 6. The ipsilateral graft extension (not shown) having a fluid flow lumen disposed therein may be deployed with the fluid flow lumen of the graft extension sealed to and in fluid communication with a fluid flow lumen 24 of the ipsilateral leg 14 of the main graft member of a bifurcated stent graft embodiment 10. In addition, at least one contralateral graft extension 142 having a fluid flow lumen disposed therein may be deployed with the fluid flow lumen of the graft extension 142 sealed to and in fluid communication with a fluid flow lumen 28 of a contralateral leg 16 of such a main graft member. For some embodiments, the graft extensions 142 may include an interposed self-expanding stent 144 disposed between at least one outer layer and at least one inner layer of supple layers of graft material. The interposed stent 144 disposed between the outer layer and inner layer of graft material may be formed from an elongate resilient element 146 helically wound with a plurality of longitudinally spaced turns into an open tubular configuration. For some embodiments, the interposed stent 142 is may include a superelastic alloy such as superelastic NiTi alloy. In addition, the graft material of each graft extension may further include at least one axial zone of low permeability for some embodiments.

For some embodiments, an outside surface of a graft extension 142 may be sealed to an inside surface of the respective leg 14 or 16 of the main graft and luminal surface of a patient's vasculature 101 when the graft extension 142 is in a deployed state. Such a configuration may allow for a fluid tight conduit extending from a position proximal to the aneurysm treatment site to a position distal to the aneurysm treatment site 106. For some embodiments, the axial length of the ipsilateral and contralateral legs 14 and 16 may be sufficient to provide adequate surface area contact with outer surfaces of graft extensions 142 to provide sufficient friction to hold the graft extensions 142 in place. For some embodiments, the ipsilateral and contralateral legs 14 and 16 may have an axial length of at least about 2 cm. For some embodiments, the ipsilateral and contralateral legs may have an axial length of about 2 cm to about 6 cm, more specifically, about 3 cm to about 5 cm.

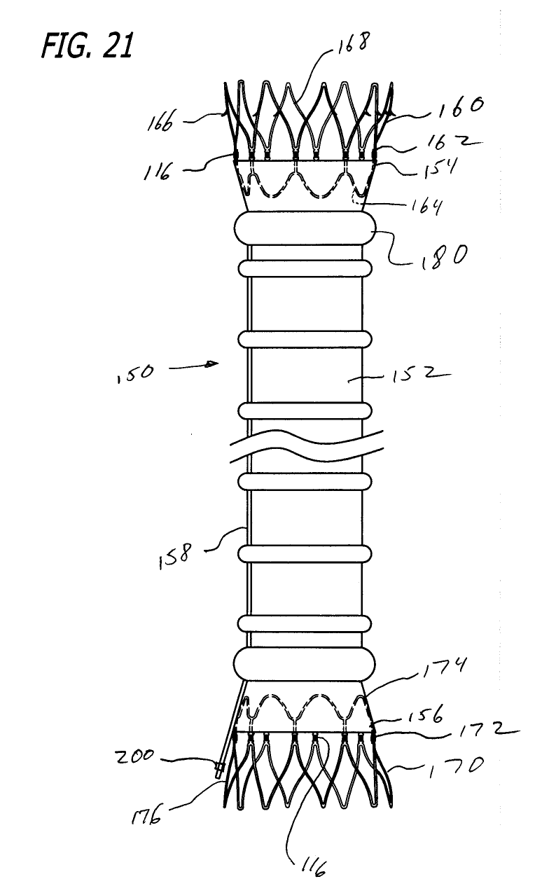

Referring to FIGS. 21-23, a tubular inflatable stent graft assembly 150 shown includes a tubular main graft member or body portion 152. The main graft 152 has a wall portion that bounds a main fluid flow lumen disposed therein. The graft body portion 152 includes a proximal end 154, a distal end 156 and an inflatable portion 158. The main body portion 152 of the stent graft 150 may include at least one flexible layer of material such as PTFE, polymer meshes, composites of same or the like. A proximal anchor member or stent 160 is disposed at the proximal end 154 of the graft body 152. The proximal anchor member 160 embodiment shown in FIG. 21 includes a single self-expanding stent member disposed at a proximal position of the stent graft. The proximal self-expanding stent member 160 may be formed from an elongate element having a generally serpentine shape with eight crowns or apices at either end for some embodiments. A distal end 162 of the proximal self-expanding stent member 160 may be mechanically coupled to a connector ring 164 which is embedded in graft material of the proximal end 154 of the main graft 152, or directly coupled to perforations in the proximal edge region of the main graft 152.

Embodiments of the connector ring 164 may be generally circular or cylindrical in shape with regular undulations about the circumference that may be substantially sinusoidal or zig-zag in shape. Some embodiments of the proximal self-expanding stent member 160 may include outwardly extending barbs 166. Such barbs 166 may be integrally formed with the struts 168 of the stent 160, having sharp tissue penetrating tips that are configured to penetrate into tissue of an inside surface of a lumen within which the proximal stent 160 is deployed in an expanded state.

A distal self-expanding member or stent 170 is disposed at the distal end 156 of the graft body and is configured to engage an interior luminal surface within the patient's vasculature 101. The distal stent member embodiment 170 shown in FIG. 21 includes a single self-expanding stent member disposed at the distal end 156 of the tubular main body portion 152 of the stent graft 152. The distal self-expanding stent member 170 may be formed from a resilient elongate element having a generally serpentine shape with eight crowns or apices at either end. A proximal end 172 of the distal self-expanding stent member 170 may be mechanically coupled to a connector ring 174 which is embedded in graft material of the distal end of the main graft 152, or directly coupled to perforations in the distal edge region of the main graft 152.

Embodiments of the distal connector ring 174 may be generally circular or cylindrical in shape with regular undulations about the circumference that may be substantially sinusoidal or zig-zag in shape. Some embodiments of the distal self-expanding stent member 170 may optionally include outwardly extending barbs 166 (not shown). Such barbs 166 may be integrally formed with the struts 176 of the stent 170, having sharp tissue penetrating tips that are configured to penetrate into tissue of an inside surface of a lumen within which the distal stent 170 is deployed in an expanded state.

Although the proximal and distal self-expanding stent members 160 and 170 are shown as including self-expanding stents, the stent members 160 and 170 may also include similar stents that are configured to be inelastically expanded with outward radial pressure as might be generated by the expansion of an expandable balloon from within either or both of the first and second stents. The connector rings 164 and 174 coupled to the stent members 160 and 170 may also be inelastically expandable for some embodiments. The self-expanding stent members 160 and 170 may be made from or include a superelastic alloy, such as NiTi alloy.

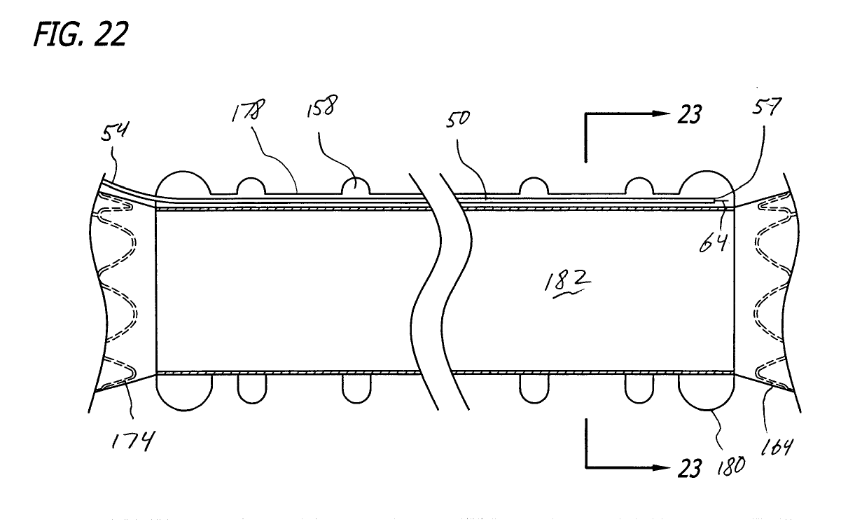

The stent graft 150 includes an optional inflation conduit 50 shown in FIG. 22 which may serve as a fill manifold for inflation of the inflatable portion 150 of the stent graft 150. The inflation conduit 50 may have some or all of the features, dimensions or materials of the inflation conduits 50 and 50′ discussed above. The inflation conduit 50 is shown disposed within the inflatable portion 158 of the stent graft 150. The inflation conduit 50 includes a distal end 54 with an inflation port 56 in fluid communication with an exterior portion of the graft body portion 152 and extending from the distal end 54 into an interior volume of the inflatable portion 158 of the stent graft 150.

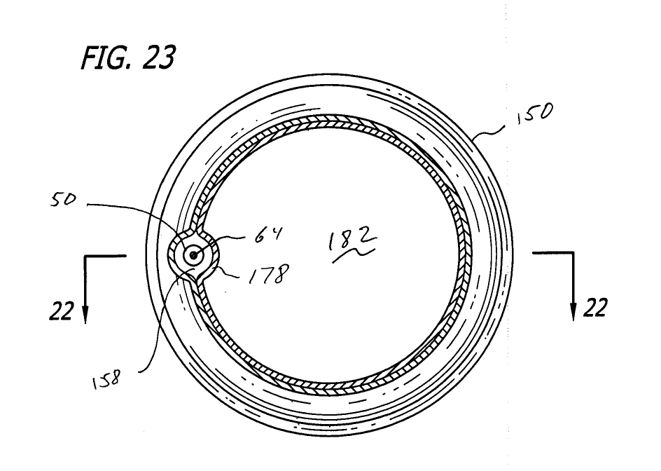

The inflation conduit 50 includes at least one outlet port 57 disposed at a desired position or desired positions within the inflatable portion 158. The inflation conduit 50 may be configured to first fill the inflatable portion 158 from the desired position or positions. The inflation conduit 50 of FIG. 23 is disposed within an interior volume of a longitudinal inflatable channel 178 of the network of inflatable channels 158 of the stent graft 150 and is configured to fill the network of inflatable channels 158 from within a proximal cuff 180 of the stent graft 150. This configuration allows the proximal cuff 180 to be filled first with fill material 13 after the proximal self-expanding member 160 has been deployed or at any other desirable time. This allows a seal to be formed between an outside surface of the proximal cuff 180 and a luminal surface of the patient's vasculature 101 at the initial inflation stage which may force a flow of blood through the main lumen 182 of the stent graft 150 and allows the stent graft main body to open sequentially in a windsock type process.

The inflation conduit 50 which is in communication between a location outside the inflatable portion 158 of the stent graft 150 and an interior volume of the inflatable portion may be disposed within any desired portion of the inflatable portion 158. The inflation conduit 50 disposed within the interior volume of the inflatable portion may include a variety of outlet port configurations. The inflation conduit 50 shown in FIG. 23 has a single outlet port 57 disposed at a proximal end 59 of the inflation conduit 50 and also includes a bead 64 disposed within an inner lumen 66 of the inflation conduit 50. Such a bead 64 may be made from a flexible but substantially incompressible material, such as solid PTFE extrusion, and may be useful for maintaining a patent lumen passage 66 through the inflation conduit 50 when the stent graft 150 and inflatable portion 158 thereof is in a constrained state prior to deployment. The outlet port 57 is disposed within an interior volume of the proximal inflatable cuff 180 disposed at a proximal end 154 of the graft body portion 152 and is configured to first inflate the proximal cuff 180 as discussed above. The inflation conduit 50 extending distally of the outlet port 57 is disposed within the longitudinal inflatable channel 178 of the inflatable portion 158 of the stent graft 150 which extends distally from the proximal inflatable cuff 180.

FIGS. 24-39 illustrate positioning and deployment method embodiments of a tubular stent graft, such as the tubular inflatable stent graft embodiment 150 shown in FIGS. 21-23 as well as others. For some embodiments, a method of deploying an inflatable endovascular stent graft 150 includes advancing a delivery catheter 184 that includes the endovascular stent graft 150 in a radially constrained state to a deployment site within a patient's vasculature 101. Once at a desired treatment site within the patient's vasculature, the endovascular stent graft 150 may be partially deployed so as to allow at least a portion of the proximal self-expanding member 160 of the endovascular stent graft 150 to radially expand. An imaging system 128 may be aligned relative to the patient's body such that an imaging axis 126 of the imaging system 128 is substantially orthogonal to a longitudinal axis 186 of a tubular main body portion 152 of the endovascular stent graft 150. The partially deployed endovascular graft 150 may then be positioned in an axial direction to a desired position within the patient's vasculature 101 to assure treatment of the treatment site and to assure proper location of the graft body 152 and proximal anchor member 160 with regard to the anatomy of the patient's vasculature 101. The proximal self-expanding member 160 of the endovascular graft 150 may then be fully deployed so as to engage an interior luminal surface within the patient's vasculature 101.

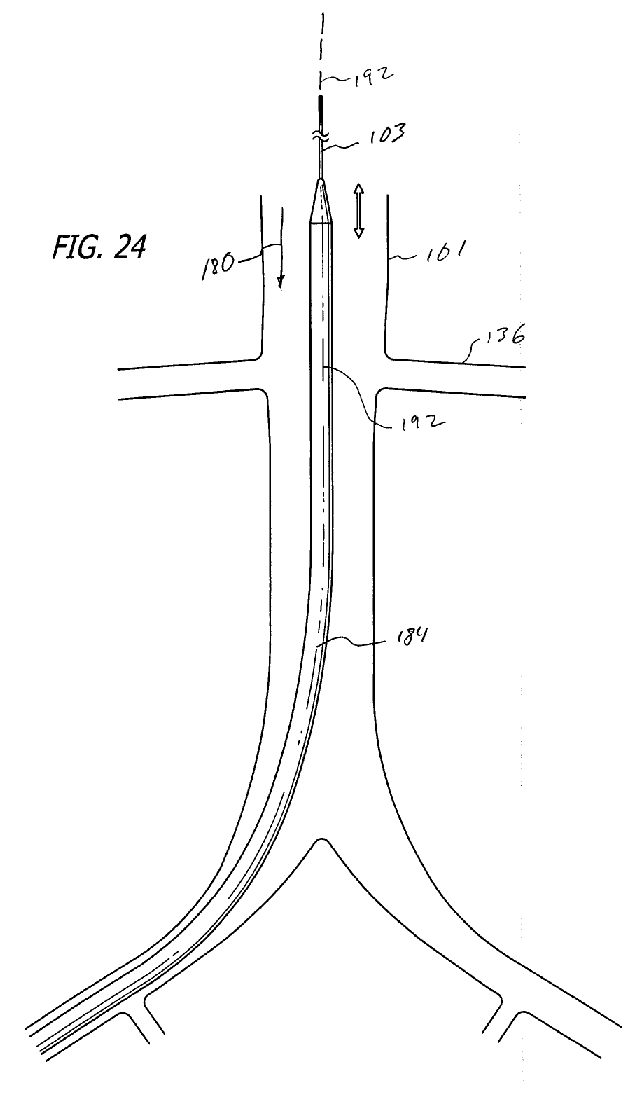

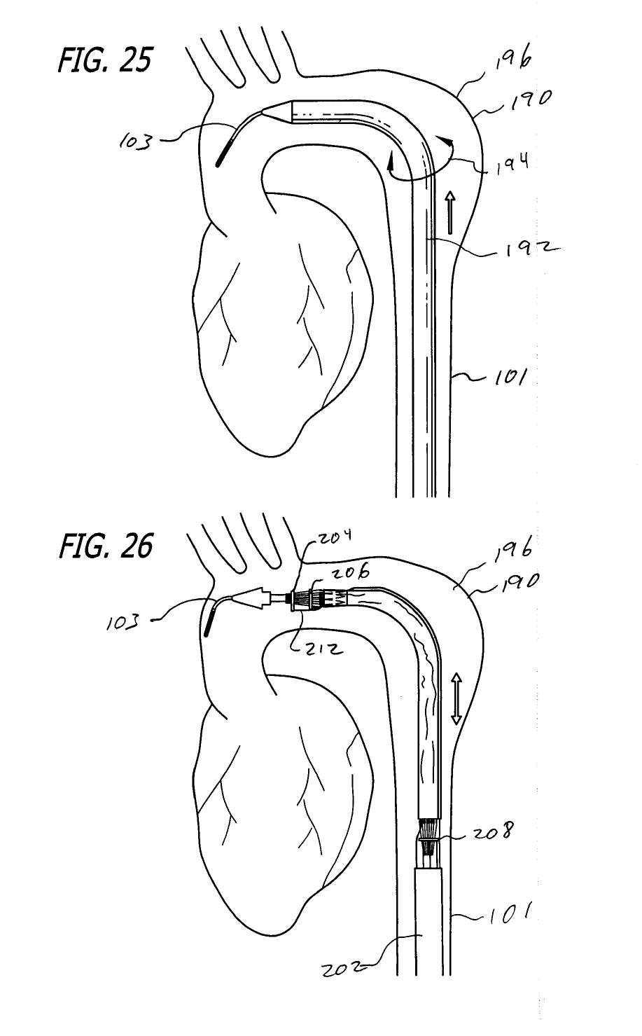

Referring to FIG. 24, a delivery catheter 184 containing the stent graft 150 is a radially constrained state is advanced within a patient's vasculature 101 with a proximal end of the stent graft 150 disposed towards a flow of blood 188 within the patient's vasculature 101. In FIG. 25, the constrained stent graft 150 is shown positioned across a thoracic aortic aneurysm treatment site 190 within a patient's vasculature 101. Such a delivery system 184 may include some or all of the features, dimensions or materials of delivery catheter systems 100 discussed above. Once at a treatment site, the delivery catheter 184 may be rotated about a longitudinal axis 192 of the delivery catheter 184, as shown by arrow 194, in order to angularly adjust the position of the stent graft 150 in the radially constrained state. For some embodiments, the delivery catheter may be angularly adjusted until the longitudinal inflatable channel 178 of the inflatable portion 158 of the endovascular stent graft 150 that extends longitudinally along a majority of a main body portion 152 of the stent graft 150 is disposed along a greater curve 196 of a vascular lumen. With the longitudinal channel 178 of the stent graft 150 disposed along a greater curve 196 of the vessel bend of the patient's vasculature 101 within which the delivery system is disposed, the stent graft may have a lower potential energy state when fully deployed and be more stable. In addition, such rotational positioning may prevent kinking of the longitudinal channel of the stent graft upon full deployment.

The rotational adjustment may also be considered in the context of disposing the longitudinal channel 178 of the stent graft 150 away from a lesser curve 198 of a bend in the patient's vasculature 101. To achieve the desired angular positioning of the stent graft 150 and delivery system 184, the stent graft 150 may include the flexible main graft body portion 152 having a proximal end 154, a distal end 156 and an inflatable portion 158 including at least one longitudinal inflation channel. In addition, the stent graft may include one or more radiopaque markers configured to distinguish circumferential rotational position of the at least one longitudinal inflation channel prior to being filled with fill material. For some embodiments, a radiopaque marker 200 maybe disposed on the distal end 54 of the inflation conduit, or any other suitable off axis position, to indicate the rotational position of the inflation conduit 50 and longitudinal channel 178 relative to the patient's vasculature 101. In some cases, visualization under fluoroscopy or the like of a relative distance of separation between the radiopaque marker 200 and the guidewire 103 maybe used to determine rotational position of the stent graft 150 relative to the patient's vasculature. This method may be particularly useful for embodiments of delivery catheters 184 that have a guidewire lumen disposed substantially in a center of the cross section of the delivery catheter 184.

Once the delivery system 184 has been positioned at the treatment site 190 an outer sheath 202 of the delivery catheter 184 may be retracted distally as shown in FIG. 26. Though the outer sheath 202 has been distally retracted with the stent graft 150 exposed, the stent graft 150 may remain in a partially constrained state with the proximal self-expanding stent member 160 restrained by a pair of proximal releasable belts 204 and 206 releasably disposed about the proximal self-expanding stent 160. The distal self-expanding stent 170 is constrained by another releasable belt 208 which is releasably disposed about the distal self-expanding member 170.

Each of the releasable belts 204, 206 and 208 may be configured to be independently released by retraction of a respective release wire. Release wires may be disposed within an end loop or loops of the releasable belts 204, 206 and 208 with the release wire holding the loops in fixed relation to each other. For this arrangement, retraction of a release wire from the end loops releases the loops to allow them to move relative to each other which in turn removes the constraint of the belt members 204, 206 and 208 about the respective stent members 160 and 170.

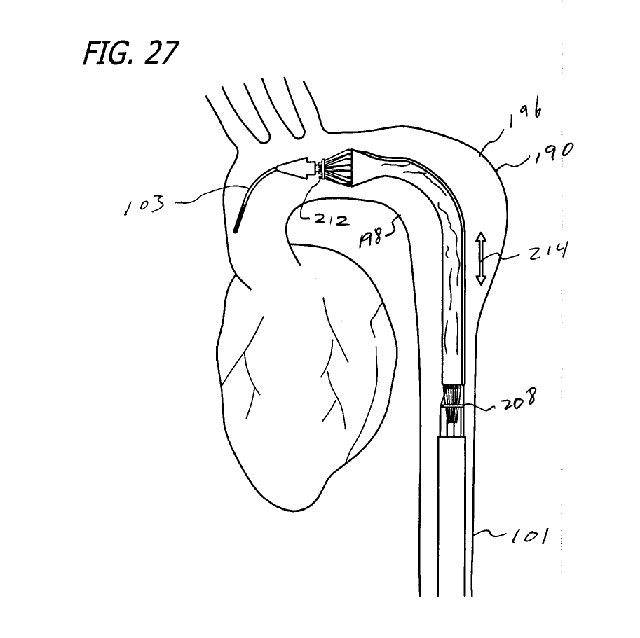

The proximal self-expanding stent member 160 may be partially deployed in some circumstances by release of one of the pair of proximal releasable belts 204 and 206. For example, the second proximal belt 206, disposed distal of a first proximal belt 204 and proximal of the proximal end of the stent graft body portion 152, may be released by retraction of a second release wire 210 so as to partially deploy the proximal self-expanding stent 160 of the stent graft 150 as illustrated in FIG. 27. In this state, the first proximal belt 204 remains undeployed with end loops or the like of the first belt 204 still held in fixed relation to each other with a first release wire 212. The first proximal belt 204 continues to radially constrain a proximal end of the proximal self-expanding stent 160.

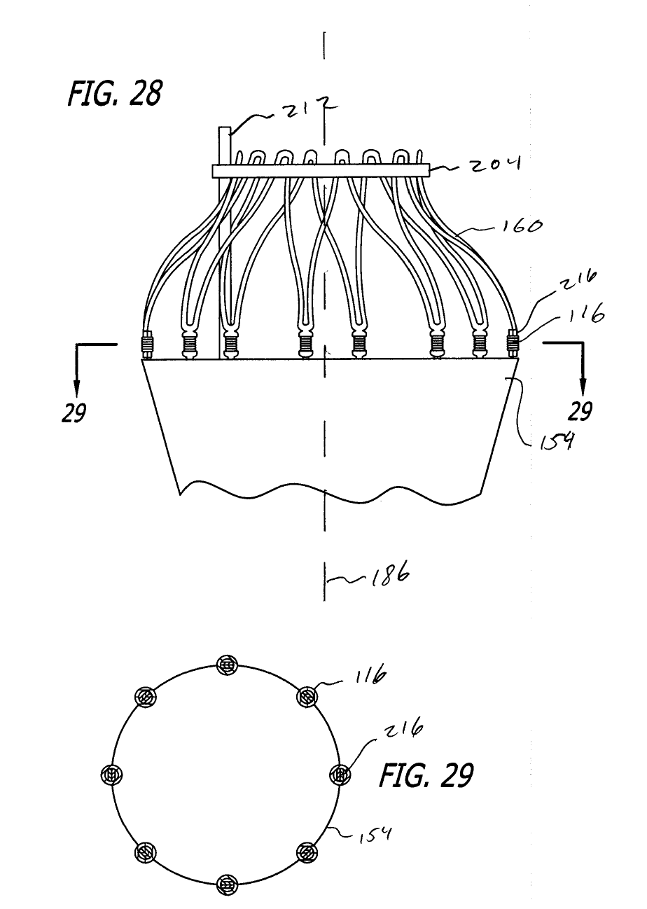

At this stage of partial deployment of the stent graft 150, finalizing the axial position of the stent graft 150 relative to the anatomy of the patient's vasculature 101 and treatment site 190 may be made as shown by arrow 214 in FIG. 27. The axial positioning may also be accomplished in some embodiments with the use of some radiopaque marker devices 116 that facilitate alignment of an imaging system 128 with the stent graft 150 and the patient's anatomy 101. FIGS. 28 and 29 illustrate in an enlarged view of a portion of the stent graft 150 with portions of the delivery catheter 184 not shown for clarity of illustration. Some radiopaque marker embodiments 116 of the stent graft 150 may be used to facilitate alignment of the stent graft 150. The radiopaque marker configuration may also be used for alignment of the imaging system 128 with respect to the stent graft 150.

For the embodiment shown, the stent graft 150 includes a tubular flexible main body portion 152, a proximal self-expanding stent member 160 and a plurality of radiopaque markers 116 circumferentially disposed about a tubular portion of the endovascular stent graft 150. The radiopaque markers 116 shown in FIGS. 28 and 29 lie in a plane that is substantially orthogonal to a longitudinal axis 186 of the tubular main body portion 152 of the stent graft 150. More particularly, for the embodiment shown, the plurality of radiopaque markers 116 is disposed about a circumference of a distal end of the proximal self-expanding stent member 160 of the stent graft 150. Even more particularly, the plurality of radiopaque markers 116 may include helically wound wire members which are disposed about connector members 216. The connector members 216 mechanically couple the proximal self-expanding stent member 160 to the connector ring 164 disposed within the proximal end 154 of the main body portion 152 of the stent graft 150.

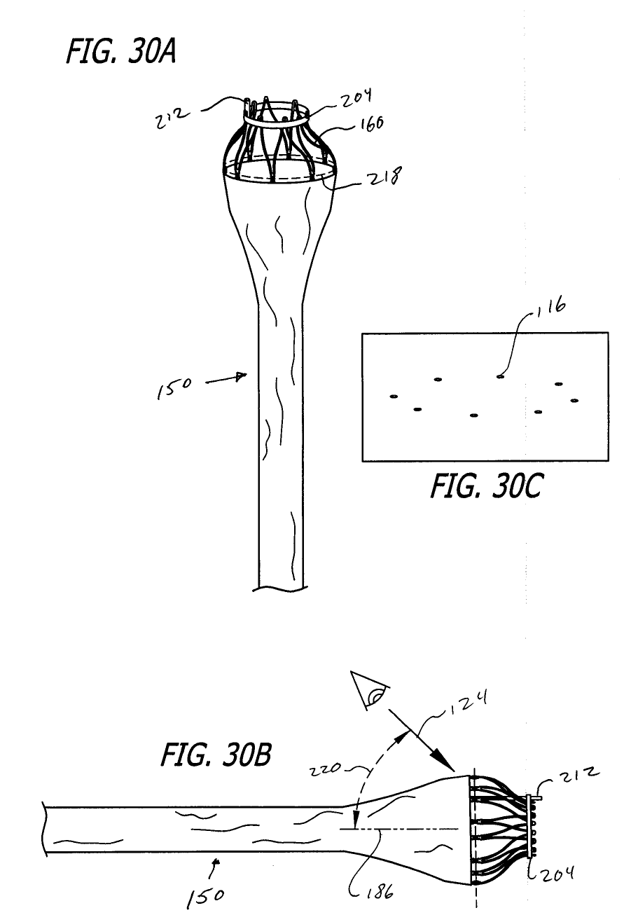

As can be seen in FIG. 29, the radiopaque markers 116 are disposed in a substantially circular arrangement about a perimeter of the stent graft 150 in a partially deployed state. This arrangement of markers 116 allows an observer of the stent graft 150 to achieve a view of the stent graft 150 which is substantially orthogonal to the longitudinal axis 186 of the stent graft 150 by aligning the markers 116 into a linear configuration. When observed from a non-orthogonal view, the radiopaque markers 116 appear as an ellipse as indicated by the dashed line 218 shown in FIG. 30A. This non-orthogonal view is further illustrated by the observer line of sight 124 relative to the longitudinal axis 186 of the stent graft 150 shown in FIG. 30B. The angle of the line of sight relative 124 to the longitudinal axis 186 of the stent graft as indicated by the arrow 220. For an orthogonal view, the angle indicated by arrow 220 would be about 90 degrees after adjustment. FIG. 30C illustrates an image of the radiopaque markers 116 that might be viewed by an observer from a non-orthogonal line of sight using an imaging system 128 that registers an image only of the radiopaque markers 116 and not the remainder of the stent graft structure.

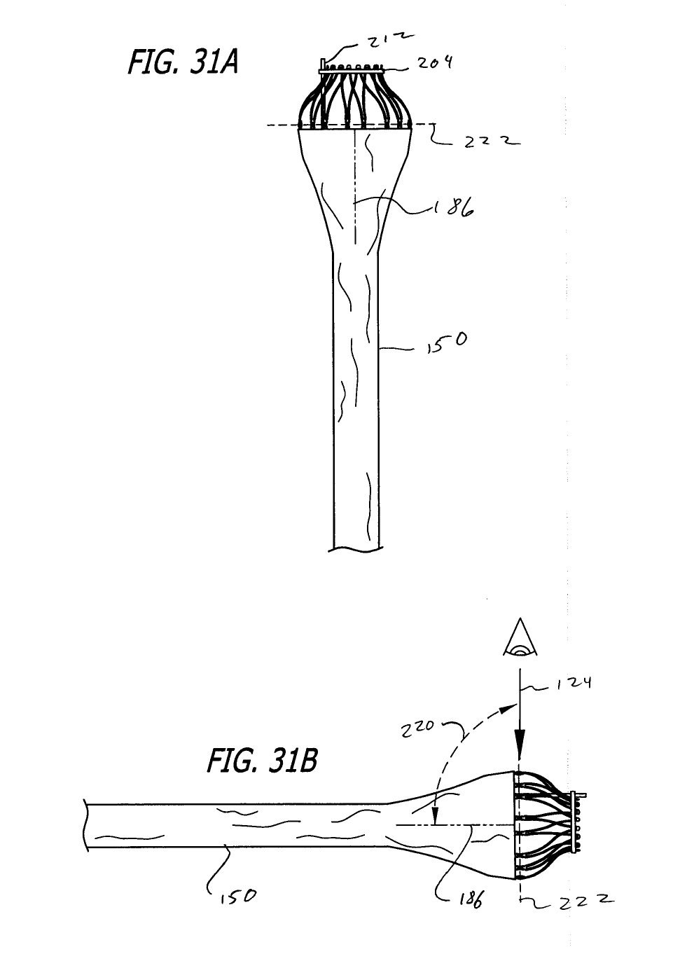

In order to achieve a substantially orthogonal view angle, the imaging system 128 used to image the stent graft 150 and patient's vasculature 101 may be adjusted in a variety of translational and angular axes 130 relative to the patient, as shown in FIG. 13 and discussed above. Such angular and translational adjustment may be made until a substantially orthogonal view angle is achieved. Such an orthogonal view is shown in FIGS. 31A and 31B wherein the image of the plurality of radiopaque markers 116 is aligned linearly to the observer as indicated by dashed line 222. As shown in these figures, the imaging system 128 aligned relative to the patient's body by aligning the plane defined by the plurality of radiopaque markers 116 substantially along the imaging axis 124 of the imaging system 128 or view angle of an observer. In such an arrangement, the radiopaque markers 116 are disposed about a circumference of a tubular portion of the stent graft 150 and lie in a plane which is substantially orthogonal to the longitudinal axis 186 of the tubular main body portion 152 of the endovascular stent graft 150. As discussed above, although an observer's eye is schematically illustrated in FIGS. 30B and 31B, the perspective illustrating the view angle 220 may be indicative of either direct observation, a view angle or imaging axis 124 of an imaging system 128 such as shown in FIG. 13, or the view angle 220 or imaging axis 124 of any other suitable imaging system. Some suitable embodiments of imaging systems for the methods discussed herein may include fluoroscopic imaging systems that use x-rays to see into a patient's body during a deployment procedure.

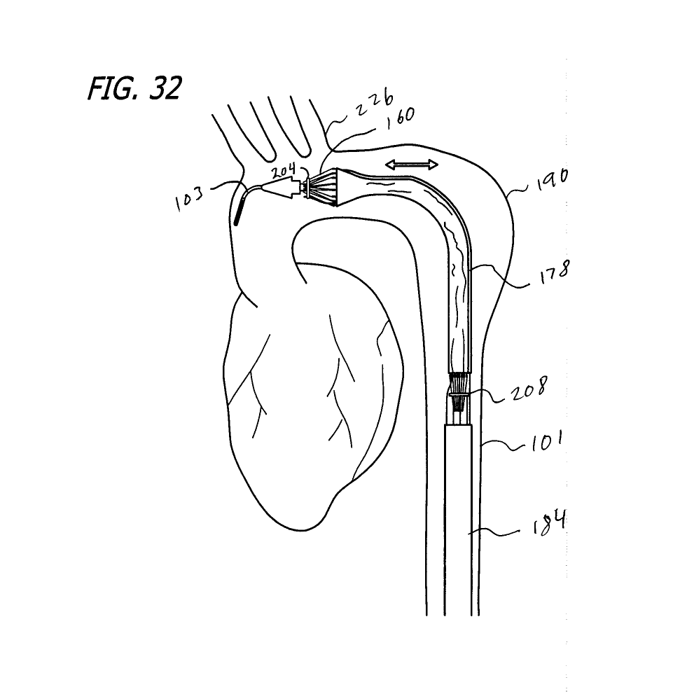

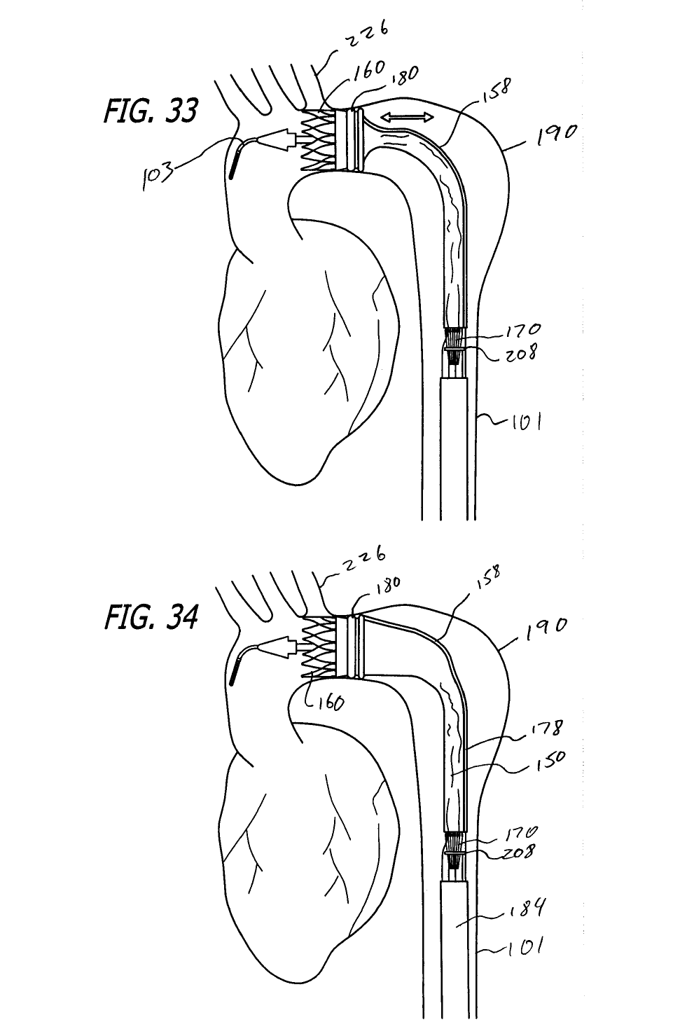

Once a substantially orthogonal view angle is achieved, an accurate axial position of the partially deployed stent graft 150 relative to the patient's vasculature 101 may be achieved. Accurate positioning may be achieved with axial movement and adjustment of the stent graft 150 by manual manipulation of a proximal portion of the delivery system (not shown) as indicated by arrow 224 in FIG. 32. As shown in FIG. 32, the stent graft 150 is positioned such that a proximal end 154 of the main body portion 152 of the stent graft 150 is aligned distal of the ostium of the left subclavian artery 226. Once the stent graft 150 in the partially radially constrained state is axially aligned, the proximal self-expanding stent member 160 may then be fully deployed so as to engage and be secured to the luminal wall or interior luminal surface of the patient's vasculature 101 as shown in FIG. 33. Once the proximal anchor member 160 is fully deployed, the inflatable portion 158 of the stent graft 150 including the network of inflatable channels may be inflated with a fill material 13. For some embodiments, the network of inflatable channels 158 may be filled from a desired site within the inflatable portion. More specifically, the inflatable portion 158 may be inflated with fill material 13 from a proximal end or portion thereof as shown in more detail in FIG. 38. Some or all of the method embodiments regarding inflation of the inflatable portion 52 of the stent graft 10 shown in FIGS. 17 and 17A and discussed above may also be applied to the inflation of the stent graft 150 illustrated in FIGS. 33 and 38.

More specifically, the fill material 13 may be dispensed or otherwise ejected under pressure from a proximal outlet port 57 of the inflation conduit 50 disposed within a proximal portion of the inflatable portion 158 of the stent graft 150. As shown, the proximal inflatable cuff 180 is full of fill material 13 and the boundary of fill material is extending distally along the longitudinal inflatable channel 178. For some embodiments, inflating a proximal portion of the inflatable portion 158 includes inflating the proximal cuff 180 first. In some circumstances, it may be useful to maintain an inner lumen opening 66 within the inflation conduit 50 prior to the inflation process with bead 64 disposed within the lumen 66 of the inflation conduit 50 as shown and discussed above. The bead 64 may be useful for maintaining at least a small luminal opening within the inner lumen 66 of the inflation conduit 50, even when the inflation conduit 50 is in a compressed or collapsed state.

Upon injection of fill material 13, the proximal inflatable cuff 180 may be inflated and expanded, as shown in FIG. 38, so as to form a seal with the luminal surface of the patient's aorta or other vascular passageway 101, as shown in FIG. 33. For some embodiments, the proximal cuff 180 of the stent graft 150 may be inflated and form a seal with a luminal surface of the patient's vasculature 101 before the inflatable portion 178 is completely filled. Once a seal is formed between an outside surface of the stent graft 150, more particularly, an outside surface of the proximal inflatable cuff 180 of the stent graft 150, and the inner luminal surface of the aorta 101, blood may begin to flow forcefully through the main lumen or conduit 182 of the stent graft 150 as shown in FIG. 34. Such a flow of blood through the main lumen 182 of the main body portion 152 may produce a windsock type action that sequentially opens the main lumen 182 in a distal direction due to the blood flow. FIG. 34 illustrates the initiation of such a windsock process whereby the blood flow or pressure within the passage or lumen 182 of the main body portion 152 opens the lumen to a full or substantially full transverse dimension or diameter. During the windsock process, the inflatable portion 178 of the stent graft 150 may continue to be inflated with fill material.

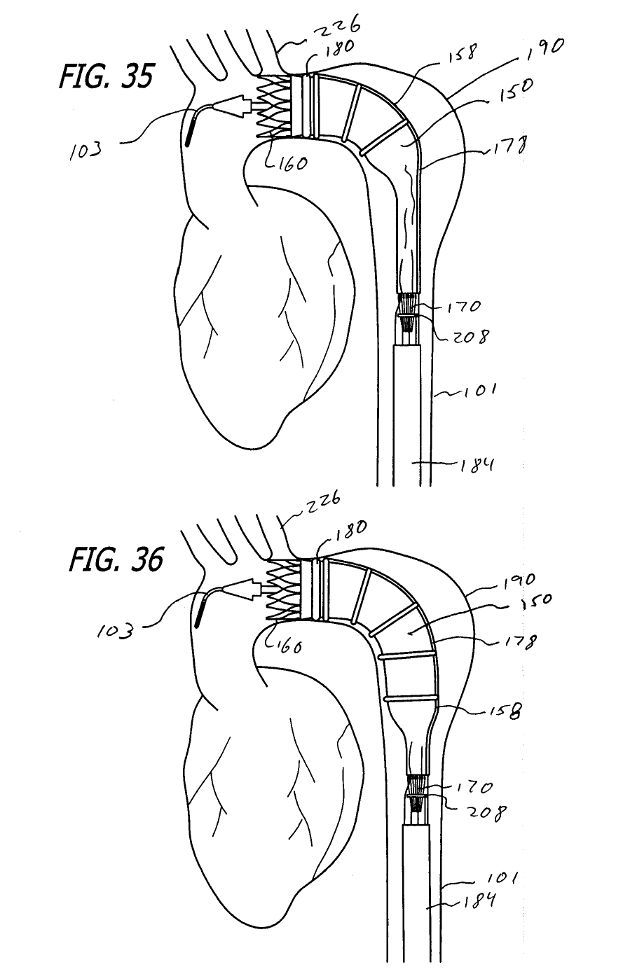

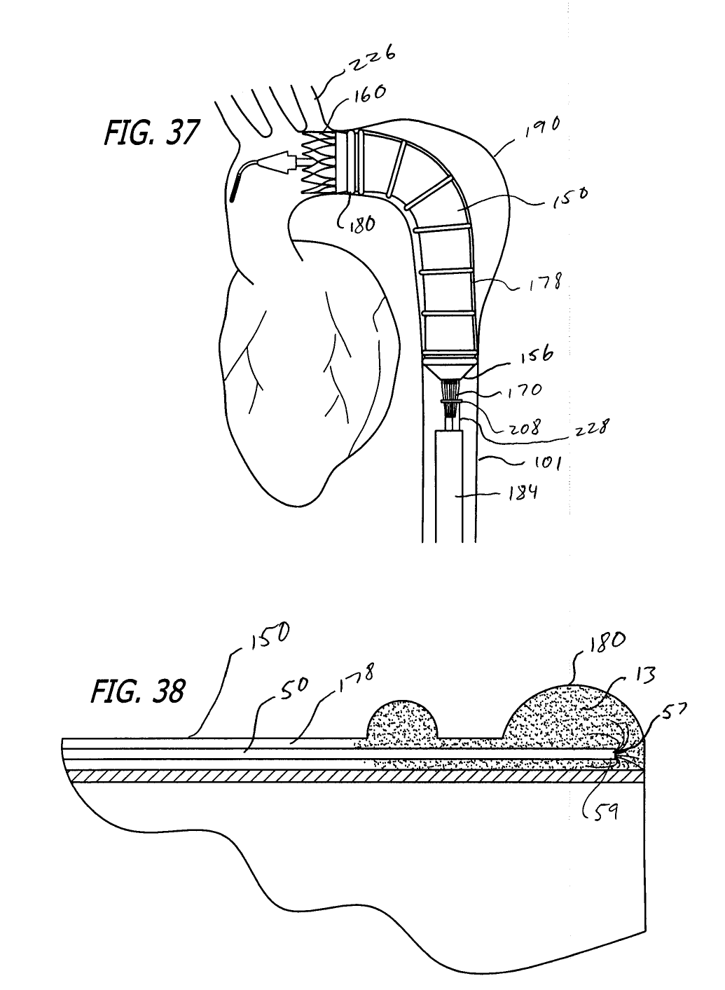

FIG. 35 illustrates the main body portion 152 of the stent graft 150 more fully filled by blood flow therethrough and with the inflatable channels 178 more fully inflated with fill material 13. For some embodiments, the windsock filling process may continue until the entire main body lumen 182 is filled or substantially filled and expanded to its full transverse dimension. FIG. 36 illustrates the stent graft 150 in an even more fully inflated state with the main lumen 182 of the tubular main graft body section 152 more fully filled with blood flow. Fill material 13 may continue to be dispensed from the inflation conduit 50 until the inflatable portion 178 of the stent graft 150 is completely filled as shown in FIG. 37. FIG. 37 illustrates the stent graft 150 with the proximal anchor member 160 completely deployed and engaged with the inner luminal wall of the patient's vasculature 101, with the inner lumen 182 of the main body portion 152 of the stent graft 150 fully filled and with the inflatable portion 178 of the stent graft 150 completely inflated. Only the distal end 156 of the stent graft 150 remains radially constrained by the distal belt 208 releasably secured around an outside surface of the distal self-expanding stent member 170. At this stage, the release wire 228 securing end loops of the distal releasable belt 208 may be retracted or otherwise deployed. This releases the end loops of the belt 208 and the radial constraint of the belt 208 about the distal self-expanding stent member 170. Thereafter, the distal stent 170 radially self-expands until the distal stent 17 completely deployed and engaged with the inner luminal wall of the patient's vasculature 101 as shown in FIG. 38.

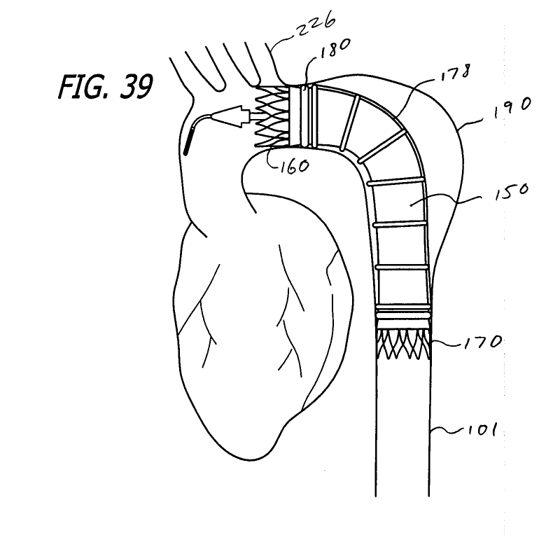

The complete inflation of the inflatable channels and cuffs 178 of the stent graft 150 may produce a structure that is sealed well against the inner luminal surface of the patient's vasculature 101, conforms to the patient's vasculature 101, is sufficiently rigid to maintain a stable structure and luminal passage 182 for blood flow. Complete inflation of the inflatable portion 158 may also produce a structure that is sufficiently flexible to maintain conformance to the patient's vasculature 101 during pulsatile cardiac cycling and blood flow. Once the stent graft 150 has been fully deployed as depicted in FIG. 39, the fill tube of the delivery catheter 184 coupled in fluid communication with the distal end 54 of the inflation conduit 50 may be uncoupled from the inflation conduit 50. In addition, the guidewire 103, fill tube and delivery catheter structure generally may then be distally retracted from the deployed stent graft 150 and the patient's vasculature 101.

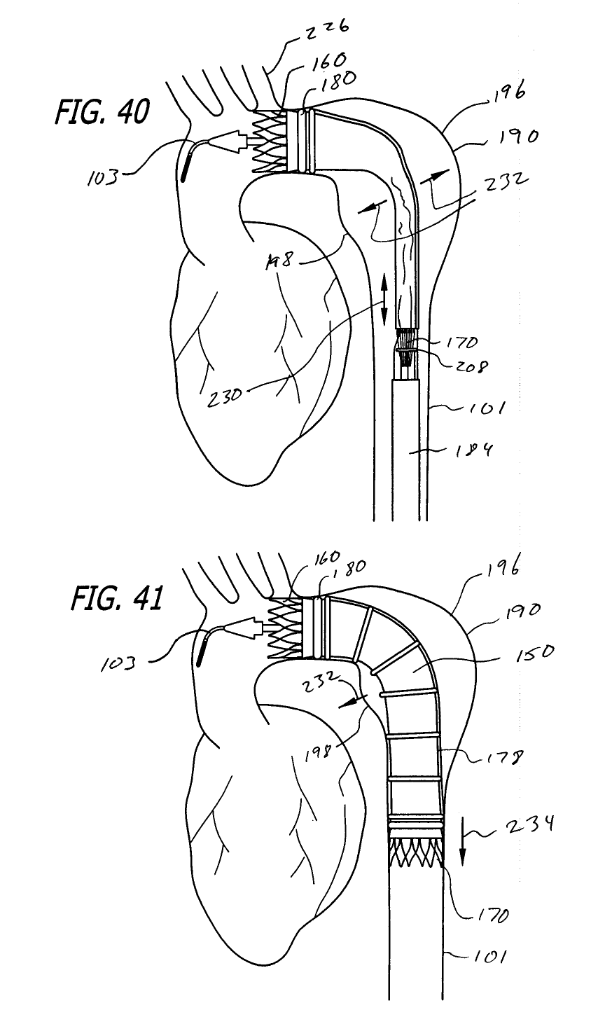

As discussed above, the deployment of the distal self-expanding stent member 170 may be one of the final actions during deployment of the stent graft 150 prior to removal of the delivery catheter 184 and components. However, for some deployment method embodiments, it may be desirable to axially adjust the position of the distal end 156 of the stent graft 150 prior to deployment of the distal stent 170 when the deployment site 190 of the patient's vasculature 101 includes a curved configuration. In particular, some deployment methods may include, as discussed above, advancing the delivery catheter 184 that includes the endovascular stent graft 150 in a radially constrained state to a deployment site 190 within a patient's vasculature 101. The proximal end of the stent graft 150 may be disposed towards a flow of blood within the patient's vasculature 101 during the advancement. The stent graft 150 may then be axially positioned relative to the deployment site 190 while in the constrained state and secured to the delivery catheter 184. The proximal self-expanding member 160 of the endovascular graft 150 may then be deployed to expand and engage an interior luminal surface the patient's vasculature 101. Additionally, at this point, a distal end 156 of the stent graft 150 may then be positioned in an axial direction, as shown by arrow 230 in FIG. 40, until a tubular main body portion 152 of the stent graft 150 achieves a desired configuration or shape within the patient's vasculature 101. Once a desired configuration or shape of the stent graft 150 is achieved, the distal self-expanding member 170 may then be deployed so as to allow the distal self-expanding member to expand and engage an interior luminal surface of the patient's vasculature 101. Deploying the distal self-expanding stent member 170 may be useful in order to fix the axial separation of the proximal self-expanding stent member 160 relative to the distal self-expanding member 170. The fixation of the axial separation may be useful in order to determine the radius of curvature and configuration of the longitudinal axis 186 of the inner lumen 182 of the main graft body 152.

For some embodiments, the distal end 156 of the stent graft 150 may be axially adjusted, as shown by arrow 230 in FIG. 40, to adjust the curvature contour of the longitudinal axis 186 of the stent graft 150 to achieve a desired flow path for blood constrained by the main lumen 182 of the stent graft 150. The longitudinal axis 186 of the stent graft 150 may be adjusted inwardly and outwardly, as indicated by arrows 232 in FIG. 40, to move the longitudinal axis 186 of the stent graft 150 and inner lumen thereof towards a greater curve 196 of the patient's vasculature 101 or towards a lesser curve 198 in the patient's vasculature 101. FIG. 41 illustrates the fully deployed stent graft 150 in a fully deployed state wherein the proximal stent member 160 has been fully deployed, the distal stent member 170 has been fully deployed and the inflatable portion 158 of the stent graft 150 has been fully inflated. For the embodiment shown in FIG. 41, the longitudinal axis 186 of the stent graft 150 is disposed towards the lesser or least curve 198 of the bend of the patient's vasculature 101 and away from the greater curve 196 of the patient's vasculature 101. With the radial position disposed along a least curve 198 of the patient's vasculature 101, the longitudinal axis 186 of the stent graft 150 may have a decreased radius of curvature relative to that which might be produced by a position disposed along a greater or greatest curve 196 of the patient's vasculature 101. Such a configuration may be achieved by applying axial tension force in a distal direction, as indicated by arrow 234, prior to deployment of the distal self-expanding member 170.

The configuration of the deployed graft 150 shown in FIG. 41 lying substantially along the lesser curve 198 of the vasculature 101 may, in some cases, include portions of the inner flow lumen 182 assuming a non-circular cross section. In some circumstances, particularly where the flexible material of the main body portion 152 of the stent graft 150 is not substantially elastic, the inner flow lumen 182 may become somewhat elliptical in cross section where the side of the stent graft 150 disposed towards the greater curve 196 is being pulled into tension towards the lesser curve. As such, it may be desirable is to deploy the stent graft 150 with the longitudinal axis of the stent graft disposed towards the greater curve 196.

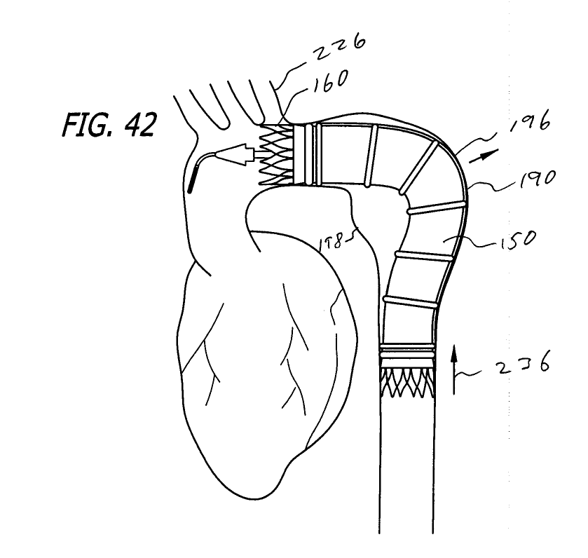

FIG. 42 illustrates the fully deployed stent graft 150 wherein the proximal stent member 160 has been fully deployed, the distal stent member 170 has been fully deployed and the inflatable portion 158 of the stent graft 150 has been fully inflated. For the embodiment shown in FIG. 42, the longitudinal axis 186 of the stent graft 150 is disposed towards the greater curve 196 of the bend of the patient's vasculature 101 and away from the lesser curve 198 of the patient's vasculature 101. With the radial position disposed along a greater curve 196 of the patient's vasculature 101, the longitudinal axis 186 of the stent graft 150 may have an increased radius of curvature relative to that which might be produced by a position disposed along a lesser or least curve 198 of the patient's vasculature 101. In some cases, such a configuration may be useful for maintaining a substantially circular cross section of the flow lumen 182 through the stent graft 150. In either case, the distal end 156 of the stent graft 150 may be axially adjusted until the longitudinal axis 186 of the inner lumen 182 of the tubular main graft body portion 152 achieves a desired radius of curvature, radial position or both within the patient's vasculature 101.

For the embodiment shown, pulling or moving the distal end 156 of the stent graft 150 in a distal direction away from the proximal end 154 of the stent graft 150 will tend to move the longitudinal axis 186 of the stent graft 150 towards the lesser or least curve 198 of the bend in the patient's aorta. Pushing the distal end 156 of the stent graft 150 towards the proximal end 154 of the stent graft 150 will tend to move the longitudinal axis 186 of the stent graft 150 more towards the greater curve 196 of the bend in the patient's vasculature 101. These adjustments may be made, in some instances, in order to adjust the radius of curvature of the flow path through the main lumen 182 of the stent graft 150, to minimize any kinking of the inner lumen 182 of the main graft body 152 or for any other applicable purpose such as those discussed above.

In some cases, the delivery catheter 184 has a longitudinal resistance to bending which causes the catheter 184 to assume a shape within the patient's vasculature that minimizes the stored energy of the catheter 184. When disposed across a bend, such as the bend shown in the patient's vasculature in FIG. 40, the delivery catheter will generally tend towards the lesser curve 198 of the vasculature to a minimum energy state. As such, when the proximal self-expanding stent member 160 is deployed, the delivery catheter 184 and remainder of the stent graft 150 secured thereto, will generally be disposed along the lesser curve 198 of the patient's vasculature. It may then be desirable to proximally advance or push the distal end of the stent graft 150 and distal self-expanding member 170 a predetermined distance prior to deploying the distal self-expanding member 170. For some embodiments, the amount of proximal advancement may be determined from the nominal transverse dimension or diameter of the main body 152 of the stent graft 150 and the magnitude of angular deflection of the stent graft 150. For example, is some instances, it may be desirable to proximally advance or displace the distal end or distal self-expanding member 170 by a distance equal to the diameter of the main body portion 152 of the stent graft 150 multiplied by the angle of deflection of the stent graft 150 in radians. As such, for the embodiment shown in FIG. 40, consider a main body portion 152 having a diameter of about 2 cm. The angle of deflection of the stent graft 150 is about 90 degrees or π/2 radians. Therefore, it may be desirable to advance the distal self-expanding member 170 by a displacement equal to 2 cm×(3.1416/2) prior to deployment of the distal self-expanding member 170. An approximation of this formula that may also be useful may include proximally advancing the distal end of the stent graft 150 by about one half the diameter of the main graft portion 152 for every 30 degrees of angular deflection prior to deployment of the distal self-expanding member 170.

For some embodiments, the axial adjustment of the distal end 156 of the stent graft 150 may be made prior to, during or after complete inflation of the inflatable portion 158 of the stent graft 150 during deployment. For some embodiments, an interior volume of an inflatable portion 158 of the endovascular stent graft 150 may be at least partially inflated from a desired location within an interior volume of the inflatable portion 158 with a fill material 13 after full deployment of the proximal stent member 160 and before deployment of the distal stent member 170.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A method of deploying an inflatable endovascular stent graft, comprising:
advancing a delivery catheter that includes the endovascular stent graft in a radially constrained state to a deployment site within a patient's vasculature;
partially deploying the endovascular graft so as to allow at least a portion of a proximal self-expanding member of the endovascular graft to radially expand;
aligning an imaging system relative to the patient's body such that an imaging axis of the imaging system is substantially orthogonal to a longitudinal axis of a tubular main body portion of the endovascular stent graft;
positioning the partially deployed endovascular graft in an axial direction to a desired position within the patient's vasculature; and
fully deploying the proximal self-expanding member of the endovascular graft so as to engage an interior luminal surface within the patient's vasculature.

2. The method of claim 1 wherein the imaging system is aligned relative to the patient's body by aligning a plane defined by a plurality of radiopaque markers substantially along the imaging axis of the imaging system, wherein the radiopaque markers are disposed about a circumference of a tubular portion of the stent graft and lie in a plane which is substantially orthogonal to a longitudinal axis of the tubular main body portion of the endovascular stent graft.

3. A method of deploying an inflatable endovascular stent graft, comprising:
advancing a delivery catheter that includes the endovascular stent graft in a radially constrained state to a deployment site within a patient's vasculature;
partially deploying the endovascular graft so as to allow at least a portion of a proximal self-expanding member of the endovascular graft to radially expand;
aligning an imaging system relative to the patient's body such that an imaging axis of the imaging system is substantially orthogonal to a longitudinal axis of a tubular main body portion of the endovascular stent graft;
positioning the partially deployed endovascular graft in an axial direction to a desired position within the patient's vasculature;
fully deploying the proximal self-expanding member of the endovascular graft so as to engage an interior luminal surface within the patient's vasculature; and
inflating an inflatable portion of the endovascular stent graft with a fill material.

4. The method of claim 3 wherein the imaging system is aligned relative to the patient's body by aligning a plane defined by a plurality of radiopaque markers substantially along the imaging axis of the imaging system, wherein the radiopaque markers are disposed about a circumference of a tubular portion of the stent graft and lie in a plane which is substantially orthogonal to a longitudinal axis of the tubular main body portion of the endovascular stent graft.

5. The method of claim 4 wherein the plurality of radiopaque markers are disposed about a circumference of the proximal self-expanding member of the stent graft.

6. The method of claim 4 wherein the plurality of radiopaque markers are disposed about a circumference of connector members of the proximal self-expanding member of the stent graft.

7. The method of claim 3 further comprising deploying a distal self-expanding member of the endovascular stent graft so as to engage an interior luminal surface within the patient's vasculature.

8. The method of claim 3 wherein the stent graft comprises a bifurcated AAA stent graft including an ipsilateral leg and a contralateral leg and further comprising deploying an ipsilateral stent graft extension into the ipsilateral leg of the bifurcated stent graft and deploying a contralateral stent graft extension into the contralateral leg of the bifurcated stent graft.

9. An endovascular stent graft, comprising:
a tubular flexible main body portion;
a proximal self-expanding stent member; and
a plurality of radiopaque markers circumferentially disposed about a tubular portion of the endovascular stent graft and lying in a plane that is substantially orthogonal to a longitudinal axis of the tubular main body portion.

10. The stent graft of claim 9 wherein the plurality of radiopaque markers are disposed about a circumference of the proximal self-expanding member of the stent graft.

11. The stent graft of claim 9 wherein the plurality of radiopaque markers are disposed about on connector members of the proximal self-expanding member of the stent graft.

12. The stent graft of claim 9 wherein the flexible graft body portion comprises at least one flexible layer of material.

13. The stent graft of claim 12 wherein the flexible layer of material comprises PTFE.

14. The stent graft of claim 9 wherein the proximal stent member comprises a substantially tubular self-expanding stent including a plurality of struts connected in a zig-zag configuration.

15. The stent graft of claim 14 wherein the proximal stent member comprises a superelastic alloy.

16. An inflatable endovascular stent graft, comprising:
a tubular flexible main body portion;
a proximal self-expanding stent member;
a proximal inflatable cuff; and
a plurality of radiopaque markers circumferentially disposed about a tubular portion of the endovascular stent graft and lying in a plane that is substantially orthogonal to a longitudinal axis of the tubular main body portion.

17. The stent graft of claim 16 wherein the plurality of radiopaque markers are disposed about a circumference of the proximal self-expanding member of the stent graft.

18. The stent graft of claim 16 wherein the plurality of radiopaque markers are disposed on connector members of the proximal self-expanding member of the stent graft.

19. The stent graft of claim 16 further comprising a distal self-expanding member of the endovascular stent graft disposed at a distal end of the tubular main body portion and configured to engage an interior luminal surface within the patient's vasculature.

20. The stent graft of claim 16 wherein the stent graft comprises a bifurcated stent graft and includes an ipsilateral leg including an inner lumen in fluid communication with an inner lumen of the tubular main body portion and a contralateral leg having an inner lumen in fluid communication with the inner lumen of the main body portion.

21. The stent graft of claim 16 wherein the flexible graft body portion comprises at least one flexible layer of material.

22. The stent graft of claim 21 wherein the flexible layer of material comprises PTFE.

23. The stent graft of claim 16 wherein the proximal stent member comprises a substantially tubular self-expanding stent including a plurality of struts connected in a zig-zag configuration.

24. The stent graft of claim 23 wherein the proximal stent member comprises a superelastic alloy.

25. A method of deploying an inflatable endovascular stent graft, comprising:
advancing a delivery catheter that includes the endovascular stent graft in a radially constrained state to a deployment site within a patient's vasculature;
rotating the delivery catheter about a longitudinal axis of the delivery catheter until a longitudinal inflatable channel of an inflatable portion of the endovascular stent graft that extends longitudinally along a main body portion of the stent graft is disposed along a greater curve of a vascular lumen of the patient's vasculature within which the delivery system is disposed; and
deploying the stent graft at the deployment site with the longitudinal inflatable channel disposed along the greater curve of the vascular lumen and inflating an inflatable portion including the longitudinal inflatable channel of the endovascular stent graft.

26. The method of claim 25 further comprising retracting an outer sheath of the delivery catheter after rotating the delivery catheter about a longitudinal axis of the delivery catheter until a longitudinal inflatable channel of the endovascular stent graft that extends longitudinally along a main body portion of the stent graft is disposed along a greater curve of a vascular lumen of the patient's vasculature.

27. A method of deploying an inflatable endovascular stent graft, comprising:
advancing a delivery catheter that includes the endovascular stent graft in a radially constrained state to a deployment site within a patient's vasculature;
rotating the delivery catheter about a longitudinal axis of the delivery catheter until a longitudinal inflatable channel of the endovascular stent graft that extends longitudinally along a main body portion of the stent graft is disposed along a greater curve of a vascular lumen of the patient's vasculature within which the delivery system is disposed;
partially deploying the endovascular stent graft so as to allow at least a portion of a proximal self-expanding member of the endovascular graft to radially expand;
positioning the partially deployed endovascular graft in an axial direction to a desired position within the patient's vasculature;
fully deploying the self-expanding member of the endovascular graft so as to allow the proximal self-expanding member of the endovascular graft to expand and engage an inner luminal surface of the patient's vasculature; and
inflating an inflatable portion of the endovascular stent graft including the longitudinal inflatable channel with a fill material.

28. The method of claim 27 further comprising aligning an imaging system relative to the patient's body after partial deployment of the proximal self-expanding member such that an imaging axis of the imaging system is substantially orthogonal to a longitudinal axis of the tubular main body portion of the endovascular stent graft.

29. The method of claim 28 wherein the imaging system is aligned relative to the patient's body by aligning a plane defined by a plurality of radiopaque markers substantially along the imaging axis of the imaging system, wherein the radiopaque marker are disposed on the self-expanding member of the stent graft and lie in a plane which is substantially orthogonal to a longitudinal axis of the tubular main body portion of the endovascular stent graft.

30. An endovascular stent graft, comprising:
a flexible graft body portion including a proximal end, a distal end, and an inflatable portion including at least one longitudinal inflation channel;
a self-expanding stent member secured to the proximal end of the graft body portion; and
one or more radiopaque markers configured to distinguish circumferential rotational position of the at least one longitudinal inflation channel prior to being filled with fill material.

31. A method of deploying an inflatable endovascular stent graft, comprising:
advancing a delivery catheter that includes the endovascular stent graft in a radially constrained state to a deployment site within a patient's vasculature with a proximal end of the stent graft disposed towards a flow of blood within the patient's vasculature;
axially positioning the stent graft in the constrained state relative to the deployment site;
deploying a proximal self-expanding member of the endovascular graft to expand and engage an interior luminal surface the patient's vasculature;
positioning a distal end of the stent graft in an axial direction until a tubular main body portion of the stent graft achieves a desired configuration; and
deploying a distal self-expanding member so as to allow the distal self-expanding member to expand and engage an interior luminal surface of the patient's vasculature.

32. The method of claim 31 further comprising aligning an imaging system relative to the patient's body such that an imaging axis of the imaging system is substantially orthogonal to a longitudinal axis of a tubular main body portion of the endovascular stent graft prior to axially positioning the stent graft.

33. The method of claim 31 further comprising inflating an inflatable portion of the endovascular stent graft with a fill material after the proximal self-expanding member has been deployed.

34. The method of claim 31 wherein deploying the proximal self-expanding member comprises:
partially deploying the endovascular graft allowing at least a portion of a proximal self-expanding member of the endovascular graft to radially expand;
positioning the partially deployed endovascular graft in an axial direction to a desired position within the patient's vasculature; and
fully deploying the proximal self-expanding member of the endovascular graft so as to allow the proximal self-expanding member to expand and engage an interior luminal surface the patient's vasculature.

35. The method of claim 32 wherein the imaging system is aligned relative to the patient's body by aligning a plane defined by a plurality of radiopaque markers substantially along the imaging axis of the imaging system, wherein the radiopaque markers are disposed on the self-expanding member of the stent graft and lie in a plane which is substantially orthogonal to a longitudinal axis of the tubular main body portion of the endovascular stent graft.

36. The method of claim 31 wherein the deployment site of the patient's vasculature comprises a curved configuration and wherein the distal end of the stent graft is positioned in an axial orientation until the tubular main body portion of the stent graft achieves a desired radius of curvature and radial position within the patient's vasculature.

37. The method of claim 36 wherein the desired radial position within the patient's vasculature comprises a radial position disposed along a greater curve of the patient's vasculature.

38. The method of claim 36 wherein the desired radial position within the patient's vasculature comprises a radial position disposed along a least curve of the patient's vasculature.

39. A method of deploying an inflatable endovascular stent graft, comprising:
advancing a delivery catheter that includes the endovascular stent graft in a radially constrained state to a deployment site within a patient's vasculature with a proximal end of the stent graft disposed towards a flow of blood within the patient's vasculature;
partially deploying the endovascular graft so as to allow at least a portion of a proximal self-expanding member of the endovascular graft to radially expand;
aligning an imaging system relative to the patient's body such that an imaging axis of the imaging system is substantially orthogonal to a longitudinal axis of a tubular main body portion of the endovascular stent graft;
positioning the partially deployed endovascular graft in an axial direction to a desired position within the patient's vasculature;
fully deploying the proximal self-expanding member of the endovascular graft so as to allow the proximal self-expanding member to expand and engage an interior luminal surface the patient's vasculature;
inflating an inflatable portion of the endovascular stent graft with a fill material;
positioning a distal end of the stent graft in an axial orientation until a tubular main body portion of the stent graft achieves a desired configuration; and
deploying a distal self-expanding member so as to allow the distal self-expanding member to expand and engage an interior luminal surface of the patient's vasculature.

40. The method of claim 39 wherein the imaging system is aligned relative to the patient's body by aligning a plane defined by a plurality of radiopaque markers substantially along the imaging axis of the imaging system, wherein the radiopaque markers are disposed on the self-expanding member of the stent graft and lie in a plane which is substantially orthogonal to a longitudinal axis of the tubular main body portion of the endovascular stent graft.

41. The method of claim 39 wherein the deployment site of the patient's vasculature comprises a curved configuration and wherein the distal end of the stent graft is positioned in an axial orientation until the tubular main body portion of the stent graft achieves a desired radius of curvature and radial position within the patient's vasculature.

42. The method of claim 41 wherein the desired radial position within the patient's vasculature comprises a radial position disposed along a greater curve of the patient's vasculature.

43. The method of claim 41 wherein the desired radial position within the patient's vasculature comprises a radial position disposed along a least curve of the patient's vasculature.

44. A method of deploying an inflatable endovascular stent graft, comprising:
advancing a delivery catheter that includes the inflatable endovascular stent graft in a radially constrained state to a deployment site within a patient's vasculature with a proximal end of the stent graft disposed towards a flow of blood within the patient's vasculature;
deploying a proximal self-expanding member of the endovascular graft so as to allow the proximal self-expanding member to expand and engage an interior luminal surface the patient's vasculature;
at least partially inflating an interior volume of an inflatable portion of the endovascular stent graft from a desired location within an interior volume of the inflatable portion with a fill material;
axially positioning a distal end of the stent graft such that a tubular main body portion of the stent graft achieves a desired deployed configuration; and
deploying a distal self-expanding member of the stent graft so as to allow the distal self-expanding member to expand and engage an interior luminal surface of the patient's vasculature.

45. The method of claim 44 wherein at least partially inflating the inflatable portion of the endovascular stent graft from a desired portion of the inflatable portion comprises inflating through an inflation conduit, the inflatable conduit being disposed within the interior volume the inflatable portion and extending to a distal portion of the stent graft.

46. The method of claim 45 wherein the inflation conduit comprises a plurality of outlet ports along a wall of the conduit, the plurality of outlet ports being in fluid communication with an interior volume of the longitudinal inflatable channel and proximal cuff of the inflatable portion and configured in size and location so as to at least partially inflate the interior volume of the inflatable portion substantially evenly with respect to a longitudinal axis of the graft body portion.

47. The method of claim 46 wherein the outlet ports in the wall of the inflation conduit have a progressively larger area in a direction from a distal end of the inflation conduit to a proximal end of the inflation conduit and configured such that a pressure gradient of the fill material within the fill tube produces substantially even flow from each outlet port.

48. The method of claim 45 wherein the inflation conduit comprises a tubular member having a single outlet port disposed within an interior volume of the proximal cuff and configured to fill the inflatable portion from a proximal end of the inflatable portion.

49. The method of claim 44 further comprising maintaining a lumen opening within the inflation conduit prior to inflation with a bead disposed within the lumen of the inflation conduit.

50. An inflatable endovascular stent graft, comprising:
at least one self-expanding stent member;
a flexible graft body portion secured to the self-expanding member, the graft body portion including a proximal end, a distal end and an inflatable portion; and
an inflation conduit which extends into an interior volume of the inflatable portion and which includes at least one outlet port disposed at a desired position or desired positions within the inflatable portion and which is configured to emit fill material injected into the inflation conduit into an interior volume of the inflatable portion from the desired position or positions of the at least one outlet port.

51. The stent graft of claim 50 wherein the inflatable portion comprises a proximal inflatable cuff disposed at a proximal end of the graft body portion.

52. The stent graft of claim 51 wherein the inflatable portion comprises an inflatable channel extending distally from the proximal inflatable cuff.

53. The stent graft of claim 50 wherein the inflation conduit comprises a plurality of outlet ports along a wall of the inflation conduit, the plurality of outlet ports being in fluid communication with an interior volume of an inflation channel and proximal cuff of the graft body portion and configured in size and location so as to inflate the inflatable portion substantially evenly with respect to a longitudinal axis of the graft body portion.

54. The stent graft of claim 53 wherein the outlet ports in the wall of the inflation conduit have a progressively larger area in a direction from a distal end of the inflation conduit to a proximal end of the inflation conduit.

55. The stent graft of claim 50 wherein the inflation conduit comprises a tubular member having a single outlet port disposed within an inner volume of a proximal cuff of the graft body portion and is configured to fill the inflatable portion from a proximal end of the inflatable portion.

56. The stent graft of claim 50 wherein the flexible graft body portion comprises at least one flexible layer of material.

57. The stent graft of claim 56 wherein the flexible layer of material comprises PTFE.

58. The stent graft of claim 50 wherein the stent member comprises a substantially tubular self-expanding stent including a plurality of struts connected in a zig-zag configuration.

59. The stent graft of claim 58 wherein the stent member comprises a superelastic alloy.

60. The stent graft of claim 50 wherein the inflation conduit comprises a PTFE tube.

61. The stent graft of claim 50 further comprising a bead disposed within the lumen of the inflation conduit.

62. A method of deploying an inflatable endovascular stent graft, comprising:
advancing a delivery catheter that includes the endovascular stent graft in a radially constrained state to a deployment site within a patient's vasculature with a proximal end of the stent graft disposed towards a flow of blood within the patient's vasculature;
inflating a proximal portion of an inflatable portion of the endovascular stent graft with a fill material with the fill material flowing from a proximal portion of the inflatable portion to a distal portion of the inflatable portion.

63. The method of claim 62 further comprising inflating a proximal cuff of the stent graft and forming a seal with a luminal surface of the patient's vasculature before the inflatable portion is completely filled.

64. The method of claim 62 further comprising deploying a proximal self-expanding member of the endovascular stent graft so as to allow the proximal self-expanding member to expand and engage an interior luminal surface the patient's vasculature.

65. The method of claim 64 further comprising positioning a distal end of the stent graft in an axial position until a tubular main body portion of the stent graft achieves a desired configuration within the patient's vasculature.

66. The method of claim 65 further comprising fully deploying a distal self-expanding member so as to allow the distal self-expanding member to expand and engage an interior luminal surface of the patient's vasculature.

67. The method of claim 62 wherein inflating from a proximal portion of the inflatable portion comprises inflating through an inflation conduit, the inflatable conduit being disposed within the inflatable portion and extending to a proximal portion of the stent graft.

68. The method of claim 67 wherein the inflation conduit comprises an outlet port disposed in a proximal cuff of the stent graft and inflating a proximal portion of the inflatable portion comprises inflating the proximal cuff.

69. The method of claim 62 further comprising maintaining a lumen opening within the inflation conduit prior to inflation with a bead disposed within the lumen of the inflation conduit.

70. An inflatable endovascular stent graft, comprising:
at least one self-expanding stent member;
a flexible graft body portion secured to the self-expanding member, the graft body portion including at least one tubular portion, a proximal end a distal end and an inflatable portion; and
an inflation conduit disposed within the inflatable portion, the inflation conduit including a distal end with an inflation port in fluid communication with an exterior portion of the graft body portion and extending from the distal end into an interior volume of the inflatable portion.

71. The stent graft of claim 70 wherein the flexible graft body portion comprises at least one flexible layer of material.

72. The stent graft of claim 71 wherein the flexible layer of material comprises PTFE.

73. The stent graft of claim 70 wherein the stent member comprises a substantially tubular self-expanding stent including a plurality of struts connected in a zig-zag configuration.

74. The stent graft of claim 73 wherein the stent member comprises a superelastic alloy.

75. The stent graft of claim 70 wherein the inflation conduit comprises a PTFE tube.

76. The stent graft of claim 70 further comprising a bead disposed within the lumen of the inflation conduit.

77. An inflatable endovascular stent graft, comprising:
at least one self-expanding stent member;
a flexible graft body portion secured to the self-expanding member, the graft body portion including at least one tubular portion, a proximal end, a distal end and an inflatable portion comprising a proximal inflatable cuff disposed at the proximal end of the graft body portion and an inflatable channel extending distally from the proximal inflatable cuff; and
an inflation conduit disposed within the inflatable channel, the inflation conduit including a distal end with an inflation port in fluid communication with an exterior portion of the graft body portion and extending from the distal end through the inflatable channel and terminating with an outlet port disposed within or near an interior cavity of the proximal inflatable cuff.

78. The stent graft of claim 77 wherein the flexible graft body portion comprises at least one flexible layer of material.

79. The stent graft of claim 78 wherein the flexible layer of material comprises PTFE.

80. The stent graft of claim 77 wherein the stent member comprises a substantially tubular self-expanding stent including a plurality of struts connected in a zig-zag configuration.

81. The stent graft of claim 80 wherein the stent member comprises a superelastic alloy.

82. The stent graft of claim 77 wherein the inflation conduit comprises a PTFE tube.

83. The stent graft of claim 77 further comprising a bead disposed within the lumen of the inflation conduit.
